(19) United States
(12) Patent Application Publication
(10) Pub. No.: US 2003/0194750 A1
(43) Pub. Date: Oct. 16, 2003

(54) Methods for treatment of diseases where GSK 3-beta is desired, and methods to identify compounds usefule for that

(76) Inventors: Han Li, Yardley, PA (US); W. Joseph Thompson, Doylestown, PA (US)

Correspondence Address: Robert W. Stevenson, Cell Pathways, Inc., 702 Electronic Drive, Horsham, PA, 19044, US

(21) Appl. No.: 10/117,255

(22) Filed: Apr. 5, 2002

(51) Int. Cl. A61K 31/00; G01N 33/53; G01N 33/567; C12Q 1/48

(52) U.S. Cl. 435/7.2; 435/15; 514/1

(57) ABSTRACT

A method for selecting compounds for the treatment of diseases where GSK3β is desired includes assessing whether the compounds cause an increase in PKG activity in the tissue of interest.

### TECHNICAL FIELD

This invention relates generally to the field of medicinal chemistry, and specifically to methods of inhibiting the activity of glycogen synthase kinase 3 (“GSK3”) in non-neoplastic conditions, and to methods of identifying compounds that inhibit GSK3 via increasing protein kinase G (“PKG”) activity and inhibiting cGMP phosphodiesterase (“PDE”).

### BACKGROUND OF THE INVENTION

The cellular actions of insulin include increased glucose transport, glycogen synthesis, and lipogenesis and decreased gluconeogenesis, glycogen and fat break down. The result is reduced hepatic glucose output and increased peripheral glucose utilization. In type II diabetes, most of the intracellular actions of insulin are reduced or absent. To find ways to combat insulin resistance in type II diabetes, much research has been focused on the insulin-regulated signaling pathways that normally mediate glucose production and/or glucose utilization.

GSK3 was originally identified by its ability to phosphorylate and inactivate glycogen synthase (GS), the rate-limiting enzyme in converting glucose into glycogen. GSK3 is regulated by insulin through insulin receptor/PI3K/PKB signal cascade and directly by PKB phosphorylation. Insulin binding to insulin receptor causes the trans-phosphorylation and activation of the receptors intracellular tyrosine kinases and thus results in the activation of PI3K. Active PI3K phosphorylates and activates PKB. PKB phosphorylates Ser21 of GSK3□ and Ser9 of GSK3□ to inactivate GSK3. The phosphorylation of GS by GSK3 inactivates GS activity. Hence, when GSK 3 is inhibited, it can not phosphorylate GS. Therefore, GS remains active and synthesizes glycogen to keep the blood glucose level low.

Increased GSK3 expression in diabetic muscle has been reported and has been proposed to contribute to the impaired GS activity and skeletal muscle insulin resistance present in type 2 diabetes.

Other activities of GSK3 in a biological context include GSK3's ability to phosphorylate tau protein in vitro as described in Mandelkow and Mandelkow, Trends in Biochem Sci (1993)18:480-83; Mulot et al, FEBS Lett (1994) 349:359-64; and Lovestone et al, Curr Biol (1995)4:1077-86; and in tissue culture cells as described in Latimer et al, FEBS Lett (1995) 365:42-46. Phosphorylation of tau and polymerization of the phosphorylated tau is believed to allow formation of paired helical filaments that are characteristic of Alzheimer's disease. Thus, inhibition of GSK3 may be useful to treat or inhibit these disorders.

Thus, a number of companies have been developing GSK3 inhibitors as potential treatment for type II diabetes and Alzheimer's disease. GSK3 inhibitors also being developed to treat other neurodegenerative diseases because GSK3 regulates several key components in neurons.

The unfortunate side effect of GSK3 inhibition is through the other down-stream-signaling cascade of GSK3 relevant to carcinogenesis. GSK3 plays important role in mediating wnt singling pathways. Accordingly, inactivation of GSK3 will result in the accumulation of oncoprotein, □-catenin. As the oncology community recognizes, the accumulation of intracellular □-catenin is associated with the development of neoplasia and the progression of cancer. For example, researchers have found high levels of it in patients with neoplasias containing mutations in the APC tumor-suppressing gene in colon cancer. People with mutations in this gene at birth often develop thousands of small tumors in the lining of their colon. When it functions properly, the APC gene codes for a normal APC protein that is believed to bind to and regulate intracellular β-catenin levels via GSK3 phosphorylation. The mutated APC protein in colon cancer affects the binding of the β-catenin bound to the mutant APC protein, which change in binding has heretofore been thought to prevent the phosphorylation of β-catenin by GSK3.

Increases in intracellular B-catenin or defects in that protein have also been associated with breast cancer. A recent study from Harvard Medical School in the journal Nature Cell Biology suggests that the overexpression of a protein, Pin 1, leads to the over-accumulation of beta-catenin in breast cancer cells by preventing its phosphorylation by another protein, APC. β-catenin accumulation is also reportedly implicated in additional cancer types including prostate, desmoid, hepatocellular, kidney, medulloblastoma, melanoma, gastric ovarian and pancreatic.

Indeed, oncogenesis has been reported in GSK3 inhibitor studies, and is the main concern in GSK3 inhibitor development.

### SUMMARY OF THE INVENTION

We have discovered a new method to treat non-neoplastic disorders where GSK3 inhibition is desired. We have discovered that by activating protein kinase G (“PKG”), we can inhibit GSK3 (by phosphorylation, according to our data). At the same time, we have discovered that activating protein kinase G leads to degradation of □-catenin, also by phosphorylation according to our data.

Accordingly, PKG activation has the desirable effect of inhibiting GSK3 without the undesirable effect of increasing intracellular β-catenin.

The activation of PKG in the manner taught in this application leads to a sustained, as opposed to transitory PKG activation, that then leads to GSK3 inhibition and β-catenin degradation.

Compounds disclosed herein thus open a new way to inhibit GSK3β.

### BRIEF DESCRIPTION OF THE DRAWINGS

FIG. 1 is is a Western blot of SW480 cell lysates from drug-treated cell lysates in the absence of added cGMP, where cells were treated in culture for 48 hours with DMSO (0.03%, lanes 1 and 2), exsulind (200, 400 and 600 μM; lanes 3, 4, 5) and E4021 (0.1, 1 and 10 μM, lanes 6, 7, 8).

FIG. 2 is a Western blot of SW480 cell lysates from drug-treated cell lysates in the presence of added cGMP, where cells were treated in culture for 48 hours with DMSO (0.03%, lanes 1 and 2), exsulind (200, 400 and 600uM; lanes 3, 4, 5) and E4021 (0. 1, 1 and 10 μM, lanes 6, 7, 8).

FIG. 3 is a Western blot of SW480 cell lysates from drug-treated cell lysates where cell were treated for 48 hours with various concentrations of exisulind, compounds 38 and compound A as indicated. PKG1β indicates protein level of PKG1β and GSK3β-P-ser9 indicates the level of inactivation of GSK31β by phosphorylation.

FIG. 4 is a SDS-PAGE analysis of in vitro phosphorylation study of GSK3β by PKG. The quantitation of 32P labeling of 1 μg GSK3β by various amount of PKG as indicated (0, 48, 480 and 4800 Unit) in the presence of added cGMP was analyzed by phosphoimager and shown on the left. The right panel shows the comassieblue stained corresponding PKG and GSK3, protein in the in vitro phosphorylation reactions.

FIG. 5 is a Western blot of HMEC cell lysates from drug-treated cell lysates where cell were treated for 48 hours with various concentrations of exisulind, compounds 38 and compound A as indicated. PKG1β bands indicate protein level of PKG1β GSK3β-P-ser9 bands indicate the level of inactivation of GSK31βby phosphorylation and β-catenin bands indicate protein level of β-catenin.

FIG. 6 is a Western blot of human liver cell line (WRL68) cell lysates from drug-treated cell lysates where cell were treated for 48 hours with various concentrations of exisulind, compounds 38 and compound A as indicated. PKG1β bands indicate protein level of PKG1β GSK3β-P-ser9 bands indicate the level of inactivation of GSK31β by phosphorylation.

FIG. 7 is a Western blot of rat skeleton muscle Cell Line ( L6) cell lysates from drug-treated cell lysates where cell were treated for 48 hours with various concentrations of exisulind, compounds 38 and compound A as indicated. GSK3β-P-ser9 bands indicate the level of inactivation of GSK31β by phosphorylation.

### DETAIILED DESCRIPTION

This invention is a method to inhibit GSK3 in a non-neoplastic cell in a way that also causes the degradation and phosphorylation of β-catenin. In brief, this invention involves a method of inhibiting GSK3 by activating PKG in a sustained manner in a non-neoplastic cell. If accomplished in the manner we teach, this can be done without inhibiting COX I or II enzymes.

As we unexpectedly discovered, activated PKG inhibits GSK3. At the same time, activated PKG causes β-catenin phosphorylation, which as discussed above leads to β-catenin degradation by the ubiquitin-proteasome pathway. Thus, we have a method of inhibiting GSK3 that avoids the β-catenin accumulation associated with carcinogenesis.

One manner of activating PKG is directly. Another method is indirect, e.g., by increasing the level of cGMP in a cell. cGMP can be increased in a cell by inhibiting the cGMP phosphodiesterase(s) (“PDE”) in the cell. cGMP PDEs include PDE1, PDE2 and PDE5.

Inhibition of GSK3 is an approach for the treatment of various diseases including type II diabetes and Alzheimer's disease.

Glycogen synthase kinase 3 (GSK3) is a proline-directed serine/threonine kinase originally identified as an activity that phosphorylates glycogen synthase (see, e.g., Woodgett, Trends Biochem Sci (1991) 16:177-81). GSK3 consists of two isoforms, .alpha. and .beta., and is constitutively active in resting cells, inhibiting glycogen synthase by direct phosphorylation. Upon insulin activation, GSK3 is inactivated, thereby allowing the activation of glycogen synthase and possibly other insulin-dependent events. GSK3 activity is also inactivated by other growth factors or hormones, that, like insulin, signal through receptor tyrosine kinases (“RTKs”). Examples of such signaling molecules include IGF-1 and EGF as described in Saito et al, Biochem J (1994) 303:27-31; Welsh et al, Biochem J (1993) 294:625-29; and Cross et al, Biochem J (1994) 303:21-26. Importantly, GSK3 has been shown to phosphorylate β-catenin as described in Peifer et al, Develop Biol (1994) 166:543-56.

As is well knowm GSK3β is involved in glucose metabolism. Specifically, it inactivates glycogen synthetase, which when activated coverts glucose to glycogen. Thus, GSK3β inhibition leads to the glucose conversion to glycogen, which in the type II diabetic, can cause a lowering of blood sugar levels. With compounds and methods of this invention GSK3β inhibition can circumvent the insulin signal pathway defect in type II diabetes.

Exisulind and other non-specific cGMP PDE inhibitors have been shown to induce apoptosis in colon cancer cells through activating PKG and reducing B-catenin. In our research, we have also observed that non-specific cGMP PDE inhibitors inactivate GSK3□ by Ser9 phosphorylation on GSK3□ □the phosphorylation of Ser21 of GSK3□ and the PKG and PKB involvement are currently under study). So exisulind and its analog showed a unique property by inhibiting GSK without causing β-catenin accumulation.

We recommend that compounds that are used in invention that increase the activity of PKG and inhibit GSK3B do not increase PKB/Akt kinase activity. When PKB/Akt kinase activity is increased, that is counter-productive because apoptosis is reduced. Desirable compounds for the purposes of this invention increase PKG activity but do not substantially incease PKB/Akt kinase activity. Methods useful to ascertain PKB/Akt kinase activity are well known in the art. In fact, activators of PKB/Akt kinase are known to inhibit GSK3β.

Increasing PKG Activity

Using the PKG assay described in U.S. Pat. No. 6,130,053, the following experiments were performed to provide methodologies to increase PKG activity due either to increase in PKG expression or an increase in cGMP levels (or both) in human cells. One method to increase PKG activity in human cells is to inhibit the cGMP PDEs in the cells. We have found that there are several such PDEs present in such cells, including PDE2, PDE5 and PDE1. A compound that inhibits all these types of PDE is desirable, accordingly. Below, we provide examples of such PDE-inhibiting compounds.

Increasing PKG activity by cGMP PDE inhibition is believed to occur when cGMP levels increase in a cell as a result of cGMP PDE inhibition. The increase in cGMP causes PKG activity to increase. Other methods of increasing cGMP activity include employing

Test Procedures

Two different types of PDE inhibitors were evaluated for their effects on PKG in neoplastic cells. A non-specific cGMP PDE inhibitors, exisulind, was evaluated since it is inhibits PDE s1, 2 and 5. Also, aPDE5-specific inhibitor, E4021, was evaluated to ascertain whether PKG elevation was simply due to PDE5-specific inhibition.

To test the effect of cGMP PDE inhibition on neoplasia containing the APC mutation, SW480 colon cancer cells were employed. SW 480 is known to contain the APC mutation. About 5 million SW480 cells in RPMI 5% serum are added to each of 8 dishes:

2-10 cm dishes - - - 30 μL DMSO vehicle control (without drug),

3-10 cm dishes - - - 200 μM, 400 μM, 600 μM exisulind in DMSO, and

3-10 cm dishes - - - E4021; 0.1 μM, 1 μM and 10 μM in DMSO.

The dishes are incubated for 48 hrs at 37° C. in 5% CO₂ incubator.

The liquid media are aspirated from the dishes (the cells will attach themselves to the dishes). The attached cells are washed in each dish with cold PBS, and 200 μL cell lysis buffer (i.e., 50 mM Tris-HCl, 1% NP-40, 150 mM NaCl, 1 mM EDTA, 1 mM Na₃VO₄, 1 mM NaF, 500 μM IBMX with proteinase inhibitors) is added to each dish. Immediately after the cell lysis buffer is added, the lysed cells are collected by scraping the cells off each dish. The cell lysate from each dish is transferred to a microfuge tube, and the microfuge tubes are incubated at 4° C. for 15 minutes while gently agitating the microfuge tubes to allow the cells to lyse completely. After lysis is complete, the microfuge tubes are centrifuged full speed (14,000 r.p.m.) for 15 minutes. The supernatant from each microfuge tube is transferred to a fresh microfuge tube.

A protein assay is then performed on the contents of each microfuge tube because the amount of total protein will be greater in the control than in the drug-treated samples, if the drug inhibits cell growth. Obviously, if the drug does work, the total protein in the drug-treated samples should be virtually the same as control. In the above situation, the control and the E-4021 microfuge tubes needed dilution to normalize them to the high-dose exisulind-treated samples (the lower dose groups of exisulind had to be normalized to the highest dose exisulind sample). Thus, after the protein assays are performed, the total protein concentration of the various samples must be normalized (e.g., by dilution).

For each drug concentration and control, two PKG assays are performed, one with added cGMP, and one without added cGMP, as described in detail below. The reason for performing these two different PKG assays is that cGMP specifically activates PKG. When PKG activity is assayed using the novel PKG assay of this invention, one cannot ascertain whether any increase the PKG activity is due to increased cGMP in the cells (that may be caused by cGMP-specific PDE inhibition) or whether the PKG activity level is due to an increased expression of PKG protein. By determining PKG activity in the same sample both with and without added cGMP, one can ascertain whether the PKG activity increase, if any, is due to increased PKG expression. Thus, if an anti-neoplastic drug elevates PKG activity relative to control, one can establish if the drug-induced increase is due to increased PKG protein expression (as opposed to activation) in the drug-treated sample if (1) the drug-treated sample with extra cGMP exhibits greater PKG activity compared to the control sample with extra cGMP, and (2) the drug-treated sample without extra cGMP exhibits greater PKG activity relative to control.

After, parallel samples with and without added cGMP are prepared, 50 μL of each cell lysate is added to 20 μL of the PDE5/GST solid phase substrate slurry described above. For each control or drug cell lysate sample to be evaluated, the reaction is started by adding phosphorylation buffer containing 10 μCi ³²P-γ-ATP solution (200 μM ATP, 4.5 mM MgCl; 5 mM KH₂PO₄; 5 mM K₂HPO₄;) to each mixture. The resultant mixtures are incubated at 30° C. for 30 minutes. The mixtures are then centrifuged to separate the solid phase, and the supernatant is discarded. The solid phase in each tube is washed with 700 μL cold PBS. To the solid phase, Laemmli sample buffer (Bio-Rad) (30 μL) is added. The mixtures are boiled for 5 minutes, and loaded onto 7.5% SDS-PAGE. The gel is run at 150 V for one hour. The bands obtained are stained with commassie blue to visualize the 85 Kd GST-PDE5 fusion protein bands, if present. The gel is dried, and the gel is laid on x-ray film which, if the PDE5 is phosphorylated, the film will show a corresponding darkened band. The darkness of each band relates to the degree of phosphorylation.

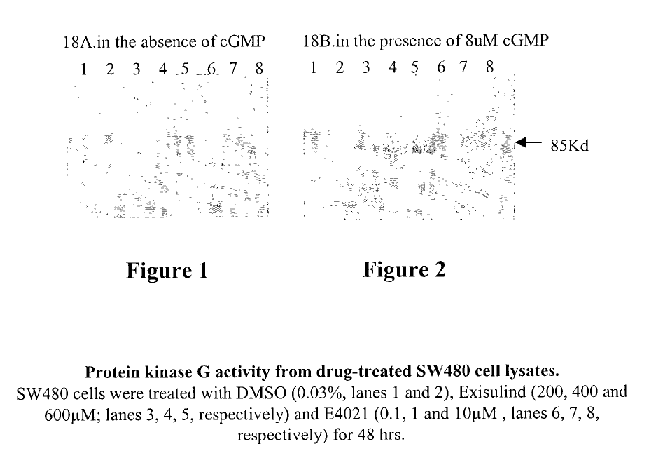

As shown in FIGS. 1 and 2, the non-specific cGMP PDE inhibitor, exisulind, causes PKG activity to increase in a dose-dependent manner in both the samples with added cGMP and without added cGMP relative to the control samples with and without extra cGMP. This is evidenced by the darker appearances of the 85 Kd bands in each of the drug-treated samples. In addition, the SW480 samples treated with exisulind show a greater PKG phosphorylation activity with added cGMP in the assay relative to the samples treated with exisulind alone (i.e. no added cGMP). Thus, the increase in PKG activity in the drug-treated samples is not due only to the activation of PKG by the increase in cellular cGMP when exisulind inhibits cGMP-specific PDE, the increase in PKG activity in neoplasia harboring the APC mutation is due to increased PKG expression as well.

Also the fact that the E4021-treated SW480 samples do not exhibit PKG activation relative to control (see FIGS. 18A and 18B) shows that the increased PKG activation caused by exisulind in cells containing the APC mutation is not simply due to specific inhibition of PDE5.

As an analytic technique for evaluating PKG activation, instead of x-ray film exposure as described above, the 85 Kd band from the SDS page can be evaluated for the degree of phosphorylation by cutting the band from the gel, and any ³²P incorporated in the removed band can be counted by scintillation (beta) counter in the ³²P window.

To test the effect of cGMP PDE inhibition on neoplasia containing the β-catenin mutation, HCT116 colon cancer cells were employed. HCT116 is known to contain the β-catenin mutation, but is known not to contain the APC mutation.

The same procedure is used to grow the HCT116 cells as is used in the SW480 procedure described above. In this experiment, only exisulind and controls were used. The exisulind-treated cells yielded PKG that was phosphorylated to a greater extent than the corresponding controls, indicating that PKG activation occurred in the drug-treated cells that is independent of the APC mutation.

Thus, for the purposes of the present invention, we refer to “reducing β-catenin” in the claims to refer to wild type and/or mutant forms of that protein.

Confirmation of Increased PKG Expression and Decreased β-Catenin

As demonstrated above, an increase in PKG expression and an increase in cGMP level, cause a sustained increase in PKG activity. This increase in PKG protein expression was further verified by relatively quantitative western blot, as described below.

SW480 cells treated with exisulind as described previously are harvested from the microfuge tubes by rinsing once with ice-cold PBS. The cells are lysed by modified RIPA buffer for 15 minutes with agitation. The cell lysate is spun down in a cold room. The supernatants are transferred to fresh microcentrifuge tubes immediately after spinning. BioRad DC Protein Assay (Temecula, Calif.) is performed to determine the protein concentrations in samples. The samples are normalized for protein concentration, as described above.

50 μg of each sample is loaded to 10% SDS gel. SDS-PAGE is performed, and the proteins then are transferred to a nitrocellulose membrane. The blotted nitrocellulose membrane are blocked in freshly prepared TBST containing 5% nonfat dry milk for one hour at room temperature with constant agitation.

A goat-anti-PKG primary antibody is diluted to the recommended concentration/dilution in fresh TBST/5% nonfat dry milk. The nitrocellulose membrane is placed in the primary antibody solution and incubated one hour at room temperature with agitation. The nitrocellulose membrane is washed three times for ten minutes each with TBST. The nitrocellulose membrane is incubated in a solution containing a secondary POD conjugated rabbit anti-goat antibody for 1 hour at room temperature with agitation. The nitrocellulose membrane is washed three times for ten minutes each time with TBST. The detection is performed by using Boehringer Mannheim BM blue POD substrate.

Exisulind causes the drop of β-catenin and the increase of PKG, which data were obtained by Western blot. SW480 cells were treated with exisulind or vehicle (0.1% DMSO) for 48 hours. 50 μg supernatant of each cell lysates were loaded to 10% SDS-gel and blotted to nitrocellulose membrane, and the membrane was probed with rabbit-anti-β-catenin and rabbit anti-PKG antibodies.

Reduction of β-Catenin Levels With PKG Activity Increase

This observation was made by culturing SW480 cells with either 200, 400 or600 μM exisulind or vehicle (0.1% DMSO). The cells are harvested 48 hours post treatment and processed for immunoblotting. Immuno-reactive protein can be detected by Western blot. Western blot analysis demonstrated that expression of β-catenin was reduced by 50% in the exisulind-treated cells as compared to control. These results indicate that β-catenin is reduced by non-specific cGMP PDE inhibitor treatment. Together with the results above establishing PKG activity increases with such treatment and the results below establishing that β-catenin is phosphorylated by PKG, these results indicate that the reduction of β-catenin in neoplastic cells is initiated by activation of PKG. Thus, using PKG activity in neoplasia as a screening tool to select compounds as anti-neoplastics is useful.

GSK3 Inactivation and PKG Activation

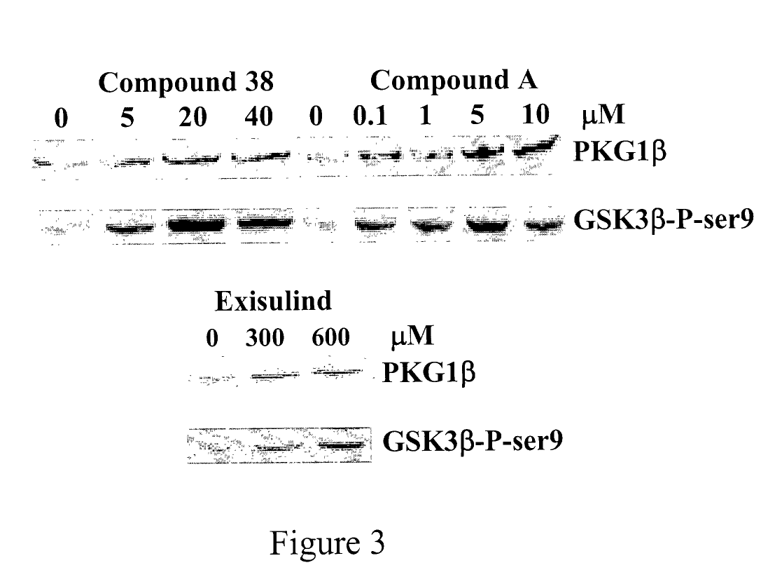

As shown in FIG. 3, SW480 cells were treated with compounds useful in this invention at various concentrations for 48 hours, then lysed with modified RIPA buffer so that the proteins could be released for assay. 50 μg protein/lane were analyzed by Western blot analysis using specific anti-PKG1β and anti-GSK3β-phospho-ser9 antibodies. The results indicate that GSK3β was inactivated in vivo by phosphorylation of its Ser9 upon treatment with compounds (as compared to control (i.e., the “0” concentrations indicated), along with PKG1 induction in a dose-dependent manner with Compound 38 (see below), Compound A (i.e., 1H-indene-3-acetamide, 5-fluoro-2-methyl-N-(phenylmethyl)-1-[(3,4,5-trimethoxyphenyl)methylene]-, (1Z)) and exisulind. Our previous data also demonstrate PKG activation in the same dose ranges.

Direct Phosphorylation of GSK3β By PKG

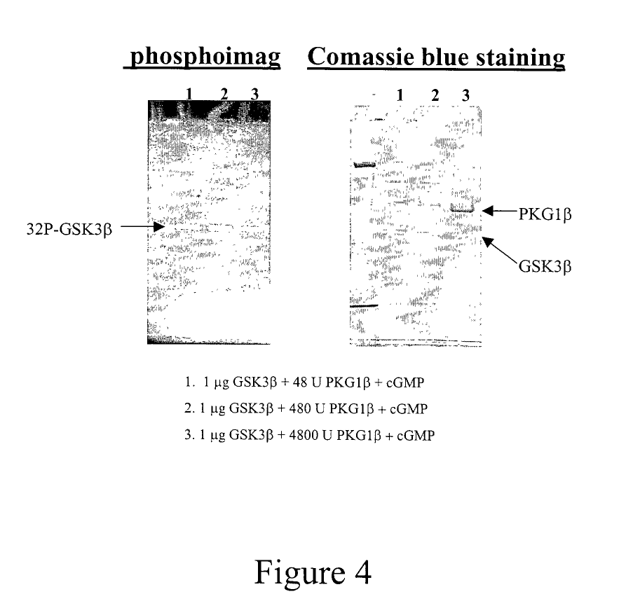

We used commercially available PKG protein to ascertain whether it phosphorylated GSK3β directly, to confirm the results above. As shown in FIG. 4, at various dilutions of PKG, it efficiently phosphorylated GSK3β in vitro.

GSK3 Inactivation and PKG Induction in Normal HMEC Cells

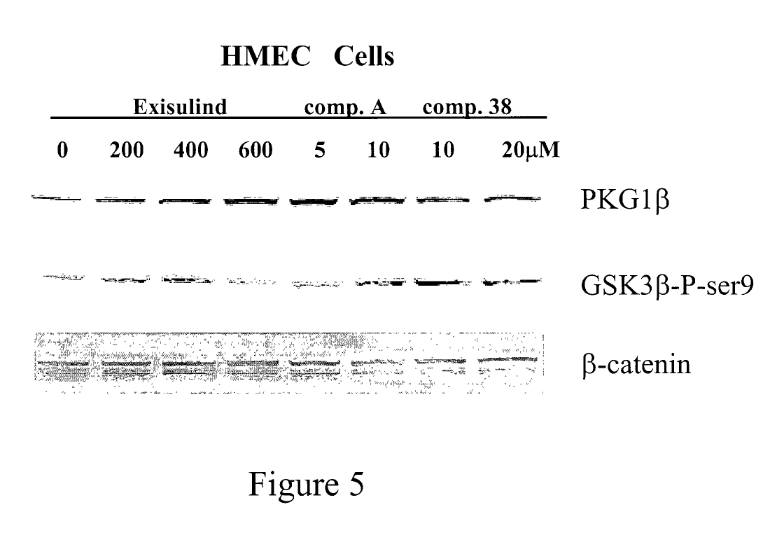

As shown in FIG. 5, we employed a non-neoplastic cell line, HMEC (a human breast line) to ascertain whether GSK3β was inactivated, PKG induced, with β-catenin levels remaining constant upon treatment with compounds useful in the practice of this invention. The results indicate that GSK3β was inactivated as evidenced by PKG phosphorylation upon treatment as compared to controls (i.e., the “0” concentrations). The results also indicate that PKG was induced in a dose-dependent manner using the several representative non-specific cGMP PDE inhibitors. Also, beneficially, β-catenin remained unchanged and did not accumulate, as would be the case if a conventional GSK3β inhibitor was employed.

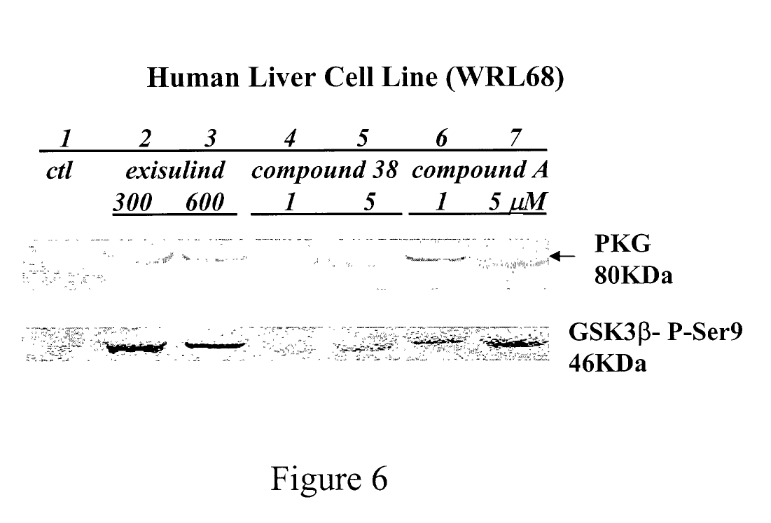

Essentially the same experiment was performed in a normal human liver cell line, WRL68. As shown in FIG. 6, the several representative non-specific cGMP PDE inhibitors caused PKG induction in a dose-dependent manner. These compounds also inactivated GSK3β in a dose-dependent manner.

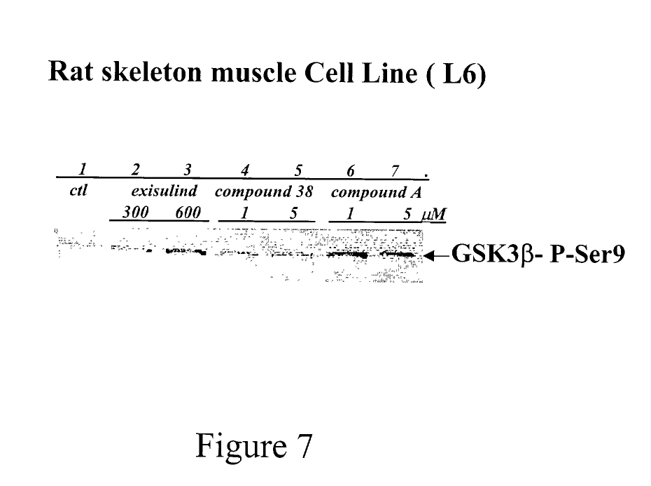

In the rat skeleton muscle (FIG. 7), GSK3β was inactivated in a dose-dependent manner with the various non-selective cGMP PDE inhibitors.

The Phosphorylation of β-Catenin By PKG

In vitro, PKG phosphorylates β-catenin. The experiment that established this involves immunoprecipitating the β-catenin-containing complex from SW480 cells (not treated with any drug) in the manner described below under “β-catenin immunoprecipitation” The immunoprecipitated complex, while still trapped on the solid phase (i.e., beads) is mixed with ³²P-γ-ATP and pure PKG (100 units). Corresponding controls with out added PKG are prepared.

The protein is released from the solid phase by SDS buffer, and the protein-containing mixture is run on a 7.5% SDS-page gel. The running of the mixture on the gel removes excess ³²P-γ-ATP from the mixture. Any ³²P-γ-ATP detected in the 93 Kd β-catenin band, therefore, is due to the phosphorylation of the β-catenin. Any increase in ³²P-γ-ATP detected in the 93 Kd β-catenin band treated with extra PKG relative to the control without extra PKG, is due to the phosphorylation of the β-catenin in the treated band by the extra PKG.

The results we obtained were that there was a noticeable increase in phosphorylation in the band treated with PKG as compared to the control, which exhibited minimal, virtually undetectable phosphorylation. This result indicates that β-catenin can be phosphorylated by PKG.

The Phosphorylation of Mutant β-Catenin By PKG

The same procedure described in the immediately preceding section was performed with HCT116 cells, which contain no APC mutation, but contain a β-catenin mutation. The results of those experiments also indicate that mutant β-catenin is phosphorylated by PKG.

Thus, for the purposes of the present invention, we refer to the phosphorylation of β-catenin in the claims to refer to the phosphorylation of wild type and/or mutant forms of that protein.

β-Catenin Precipitates With PKG

Supernatants of both SW480 and HCT116 cell lysates are prepared in the same way described above in the Western Blot experiments. The cell lysate are pre-cleared by adding 150 μl of protein A Sepharose bead slurry (50%) per 500 μg of cell lysate and incubating at 4° C. for 10 minutes on a tube shaker. The protein A beads are removed by centrifugation at 14,000×g at 4° C. for 10 minutes. The supernatant are transferred to a fresh centrifuge tube. 10 μg of the rabbit polyclonal anti-β-catenin antibody (Upstate Biotechnology, Lake Placid, N.Y.) are added to 500 μg of cell lysate. The cell lysate/antibody mixture is gently mixed for 2 hours at 4° C. on a tube shaker. The immunocomplex is captured by adding 150 μl protein A Sepharose bead slurry (75 μl packed beads) and by gently rocking the mixture on a tube shaker for overnight at 4° C. The Sepharose beads are collected by pulse centrifugation (5 seconds in the microcentrifuge at 14,000 rpm). The supernatant fraction is discarded, and the beads are washed 3 times with 800 μl ice-cold PBS buffer. The Sepharose beads are resuspended in 150 μl 2×sample buffer and mixed gently. The Sepharose beads are boiled for 5 minutes to dissociate the immunocomplexes from the beads. The beads are collected by centrifugation and SDS-PAGE is performed on the supernatant.

A Western blot is run on the supernatant, and the membrane is then probed with an rabbit anti β-catenin antibody. Then the membrane is washed 3 times for 10 minutes each with TBST to remove excess anti β-catenin antibody. A goat, anti-rabbit antibody conjugated to horseradish peroxidase is added, followed by 1 hour incubation at room temperature. When that is done, one can visualize the presence of β-catenin with an HRPO substrate. In this experiment, we could clearly visualize the presence of β-catenin.

To detect PKG on the same membrane, the anti-β-catenin antibody conjugate is first stripped from the membrane with a 62 mM tris-HCl buffer (pH 7.6) with 2% SDS and 100 μM 2β-mercaptoethanol in 55° C. water bath for 0.5 hour. The stripped membrane is then blocked in TBST with 5% non-fat dried milk for one hour at room temperature while agitating the membrane. The blocked, stripped membrane is then probed with rabbit polyclonal anti-PKG antibody (Calbiochem, LaJolla, Calif.), that is detected with goat, anti-rabbit second antibody conjugated to HRPO. The presence of PKG on the blot membrane is visualized with an HRPO substrate. In this experiment, the PKG was, in fact, visualized. Given that the only proteins on the membrane are those that immunoprecipitated with β-catenin in the cell supernatants, this result clearly establishes that PKG was physically linked to the protein complex containing the β-catenin in the cell supernatants.

The same Western blot membrane was also probed after stripping with anti-GSK3-β antibody to ascertain whether it also co-precipitated with β-catenin. In that experiment, we also detected GSK3-β on the membrane, indicating that the GSK3-β precipitated with the GSK3-β and PKG, suggesting that the three proteins may be part of the same complex. Since GSK3-β and β-catenin form part of the APC complex in normal cells, this that PKG may be part of the same complex, and may be involved in the phosphorylation of β-catenin as part of that complex.

Non-specific cGMP PDE inhbitors useful in the practice of this invention include exisulind and other compounds disclosed in U.S. Pat. Nos. 5,401,774, 6,063,818, 5,998,477, and 5,965,619. These patents are incorporated herein by reference.

When referring to an “a physiologically effective amount of an inhibitor of PDE2 and PDE5” we mean not only a single compound that inhibits those enzymes but a combination of several compounds, each of which can inhibit one or both of those enzymes. Single compounds that inhibit both enzymes are preferred. This invention, among other things, involves the inhibition of GSK3β by exposing the cells to be treated to a physiologically effective amount of an inhibitor of PDE2 and PDE5.

When referring to an “inhibitor [that] does not substantially inhibit COX I or COX II,” or “not substantially inhibiting COX” we mean that in the ordinary sense of the terms. By way of example only, if the inhibitor has an IC₅₀ for either PDE2 or PDE5 that is at least half of the IC₅₀ of COXI and/or COXII, a drug achieving the PDE IC₅₀ in the blood could be said not to substantially inhibit the COX enzymes. Preferably, the IC₅₀ for the COX enzymes is in the order of 10 fold or more higher than the IC₅₀ for PDE2/PDE5. Preferably the IC₅₀ for each of the COX enzymes is greater than about 40 μM.

When referring to “inhibiting GSK3β in mammalian cells,” we mean in vitro or in vivo. This includes therapeutic purposes.

When referring to “increasing PKG activity,” we mean either increasing catalytic phosphorylation and covalent modification of substrate, or induction of enzyme protein, resulting in enhanced catalytic phosphorylation and covalent modification.

In addition, this invention includes the use of compounds of Formula I below (as well as their pharmaceutically acceptable salts) for treating a mammal with where GSK3 inhibition is desired:

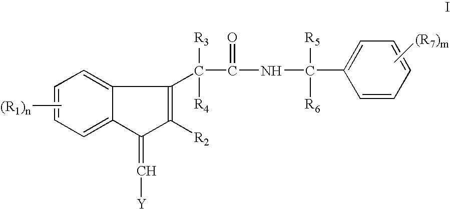

wherein R₁ is independently selected in each instance from the group consisting of hydrogen, halogen, lower alkyl, lower alkoxy, amino, lower alkylamino, di-lower alkylamino, lower alkylmercapto, lower alkyl sulfonyl, cyano, carboxamide, carboxylic acid, mercapto, sulfonic acid, xanthate and hydroxy;

R₂ is selected from the group consisting of hydrogen and lower alkyl;

R₃ is selected from the group consisting of hydrogen, halogen, amino, hydroxy, lower alkyl amino, and di-loweralkylamino;

R⁴ is hydrogen, or R₃ and R₄ together are oxygen;

R₅ and R⁶ are independently selected from the group consisting of hydrogen, lower alkyl, hydroxy-substituted lower alkyl, amino lower alkyl, lower alkylamino-lower alkyl, lower alkyl amino di-lower alkyl, lower alkyl nitrile, —CO₂H, —C(O)NH₂, and a C₂ to C₆ amino acid;

R₇ is independently selected in each instance from the group consisting of hydrogen, amino lower alkyl, lower alkoxy, lower alkyl, hydroxy, amino, lower alkyl amino, di-lower alkyl amino, halogen, —CO₂H, —SO₃H, —SO₂NH₂, and —SO₂(lower alkyl);

m and n are integers from 0 to 3 independently selected from one another;

Y is selected from the group consisting of quinolinyl, isoquinolinyl, pyridinyl, pyrimidinyl, pyrazinyl, imidazolyl, indolyl, benzimidazolyl, triazinyl, tetrazolyl, thiophenyl, furanyl, thiazolyl, pyrazolyl, or pyrrolyl, or substituted variants thereof wherein the substituents are one or two selected from the group consisting of halogen, lower alkyl, lower alkoxy, amino, lower alkylamino, di-lower alkylamino, hydroxy, —SO₂(lower alkyl) and —SO₂NH₂.

Preferred compounds of this invention for use with the methods described herein include those of Formula I where:

R₁ is selected from the group consisting of halogen, lower alkoxy, amino, hydroxy, lower alkylamino and di-loweralkylamino, preferably halogen, lower alkoxy, amino and hydroxy;

R₂ is lower alkyl;

R₃ is selected from the group consisting of hydrogen, halogen, hydroxy, amino, lower alkylamino and di-loweralkylamino, preferably, hydrogen, hydroxy and lower alkylamino;

R₅ and R₆ are independently selected from the group consisting of hydrogen, hydroxy-substituted lower alkyl, amino lower alkyl, lower alkylamino-lower alkyl, lower alkyl amino di-lower alkyl, —CO₂H, —C(O)NH₂; preferably hydrogen, hydroxy-substituted lower alkyl, lower alkyl amino di-lower alkyl, —CO₂H, and —C(O)NH₂;

R₇ is independently selected in each instance from the group consisting of hydrogen, lower alkoxy, hydroxy, amino, lower alkyl amino, di-lower alkyl amino, halogen, —CO₂H, —SO₃H, —SO₂NH₂, and —SO₂(lower alkyl); preferably hydrogen, lower alkoxy, hydroxy, amino, amino lower alkyl, halogen, —CO₂H, —SO₃H, —SO₂NH₂, and —SO₂(lower alkyl);

Preferably, at least one of the R₇ substituents is para- or ortho-located; most preferably ortho-located;

Y is selected from the group consisting of quinolinyl, isoquinolinyl, pyridinyl, pyrimidinyl and pyrazinyl or said substituted variants thereof.

Preferably, the substituents on Y are one or two selected from the group consisting of lower alkoxy, amino, lower alkylamino, di-lower alkylamino, hydroxy, —SO₂(lower alkyl) and —SO₂NH₂; most preferably lower alkoxy, di-lower alkylamino, hydroxy, —SO₂(lower alkyl) and —SO₂NH₂.

The present invention also is a method of treating a mammal where a need to inhibit GSK3 is desired by administering to a patient a pharmacologically effective amount of a pharmaceutical composition that includes a compound of Formula I, wherein R₁ through R₇ and Y are as defined above.

As used herein, the term “halo” or “halogen” refers to chloro, bromo, fluoro and iodo groups, and the term “alkyl” refers to straight, branched or cyclic alkyl groups and to substituted aryl alkyl groups. The term “lower alkyl” refers to C₁ to C₈ alkyl groups.

The term “hydroxy-substituted lower alkyl” refers to lower alkyl groups that are substituted with at least one hydroxy group, preferably no more than three hydroxy groups.

The term “—SO₂(lower alkyl)” refers to a sulfonyl group that is substituted with a lower alkyl group.

The term “lower alkoxy” refers to alkoxy groups having from 1 to 8 carbons, including straight, branched or cyclic arrangements.

The term “lower alkylmercapto” refers to a sulfide group that is substituted with a lower alkyl group; and the term “lower alkyl sulfonyl” refers to a sulfone group that is substituted with a lower alkyl group.

The term “pharmaceutically acceptable salt” refers to non-toxic acid addition salts and alkaline earth metal salts of the compounds of Formula I. The salts can be prepared in situ during the final isolation and purification of such compounds, or separately by reacting the free base or acid functions with a suitable organic acid or base, for example. Representative acid addition salts include the hydrochloride, hydrobromide, sulfate, bisulfate, acetate, valerate, oleate, palmatate, stearate, laurate, borate, benzoate, lactate, phosphate, tosylate, mesylate, citrate, maleate, fumarate, succinate, tartrate, glucoheptonate, lactobionate, lauryl sulfate salts and the like. Representative alkali and alkaline earth metal salts include the sodium, calcium, potassium and magnesium salts.

It will be appreciated that certain compounds of Formula I can possess an asymmetric carbon atom and are thus capable of existing as enantiomers. Unless otherwise specified, this invention includes such enantiomers, including any racemates. The separate enaniomers may be synthesized from chiral starting materials, or the racemates can be resolved by conventional procedures that are well known in the art of chemistry such as chiral chromatography, fractional crystallization of diastereomeric salts and the like.

Compounds of Formula I also can exist as geometrical isomers (Z and E); the Z isomer is preferred.

Compounds of this invention may be formulated into pharmaceutical compositions together with pharmaceutically acceptable carriers for oral administration in solid or liquid form, or for rectal or topical administration, although carriers for oral administration are most preferred.

Pharmaceutically acceptable carriers for oral administration include capsules, tablets, pills, powders, troches and granules. In such solid dosage forms, the carrier can comprise at least one inert diluent such as sucrose, lactose or starch. Such carriers can also comprise, as is normal practice, additional substances other than diluents, e.g., lubricating agents such as magnesium stearate. In the case of capsules, tablets, troches and pills, the carriers may also comprise buffering agents. Carriers such as tablets, pills and granules can be prepared with enteric coatings on the surfaces of the tablets, pills or granules. Alternatively, the enterically coated compound can be pressed into a tablet, pill, or granule, and the tablet, pill or granules for administration to the patient. Preferred enteric coatings include those that dissolve or disintegrate at colonic pH such as shellac or Eudraget S.

Pharmaceutically acceptable carriers include liquid dosage forms for oral administration, e.g., pharmaceutically acceptable emulsions, solutions, suspensions, syrups and elixirs containing inert diluents commonly used in the art, such as water. Besides such inert diluents, compositions can also include adjuvants such as wetting agents, emulsifying and suspending agents, and sweetening, flavoring and perfuming agents.

Pharmaceutically acceptable carriers for topical administration include DMSO, alcohol or propylene glycol and the like that can be employed with patches or other liquid-retaining material to hold the medicament in place on the skin so that the medicament will not dry out.

Pharmaceutically acceptable carriers for rectal administration are preferably suppositories that may contain, in addition to the compounds of this invention excipients such as cocoa butter or a suppository wax, or gel.

The pharmaceutically acceptable carrier and compounds of this invention are formulated into unit dosage forms for administration to a patient. The dosage levels of active ingredient (i.e., compounds of this invention) in the unit dosage may be varied so as to obtain an amount of active ingredient effective to achieve lesion-eliminating activity in accordance with the desired method of administration (i.e., oral or rectal). The selected dosage level therefore depends upon the nature of the active compound administered, the route of administration, the desired duration of treatment, and other factors. If desired, the unit dosage may be such that the daily requirement for active compound is in one dose, or divided among multiple doses for administration, e.g., two to four times per day.

The compounds of this invention can be formulated with pharmaceutically acceptable carriers into unit dosage forms in a conventional manner so that the patient in need of therapy can periodically (e.g., once or more per day) take a compound according to the methods of this invention. The exact initial dose of the compounds of this invention can be determined with reasonable experimentation. The initial dosage calculation would. also take into consideration several factors, such as the formulation and mode of administration, e.g. oral or intravenous, of the particular compound. A total daily oral dosage of about 50 mg-2.0 gr of such compounds would achieve a desired systemic circulatory concentration. As discussed below, an oral dose of about 800 mg/day has been found appropriate in mammals.

Preferably, the treatment of mammalian cells in need of GSK3β inhibition with a compound of this invention should be continuous over an extended period of time. We believe with such continuous, extended treatment, PKG activity will increase and that increase will be maintained, as opposed to being transient. By continuous, we do not mean to suggest that drug be present or taken all the time. We mean that it be present most of the time at levels sufficient to cause PKG activity to increase most of the time. By extended period of time, we are referring to a period of time at least to address at least in part the effects that a failure to inhibit GSK3β can cause. In the case of human diabetes, we believe that taking a compound such as compound 38 in unit doses several times per day for at least 3 days will begin to have the desired effect.

The pharmaceutical compositions of this invention are preferably packaged in a container (e.g., a box or bottle, or both) with suitable printed material (e.g., a package insert) containing indications and directions for use in the treatment of a disease where GSK3 inhibition is desired, etc.

There are several general schemes for producing compounds of Formula I useful in this invention. One general scheme (which has several sub-variations) involves the case where both R₃ and R₄ are both hydrogen. This first scheme is described immediately below in Scheme I. The other general scheme (which also has several sub-variations) involves the case where at least one of R₃ and R₄ is a moiety other than hydrogen but within the scope of Formula I above. This second scheme is described below as “Scheme II.”

The general scheme for preparing compounds where both R₃ and R₄ are both hydrogen is illustrated in Scheme I, which is described in part in U.S. Pat. No. 3,312,730, which is incorporated herein by reference. In Scheme I, R₁ is as defined in Formula I above. However, in Scheme I, that substituent can also be a reactive moiety (e.g. a nitro group) that later can be reacted to make a large number of other substituted indenes from the nitro-substituted indenes.

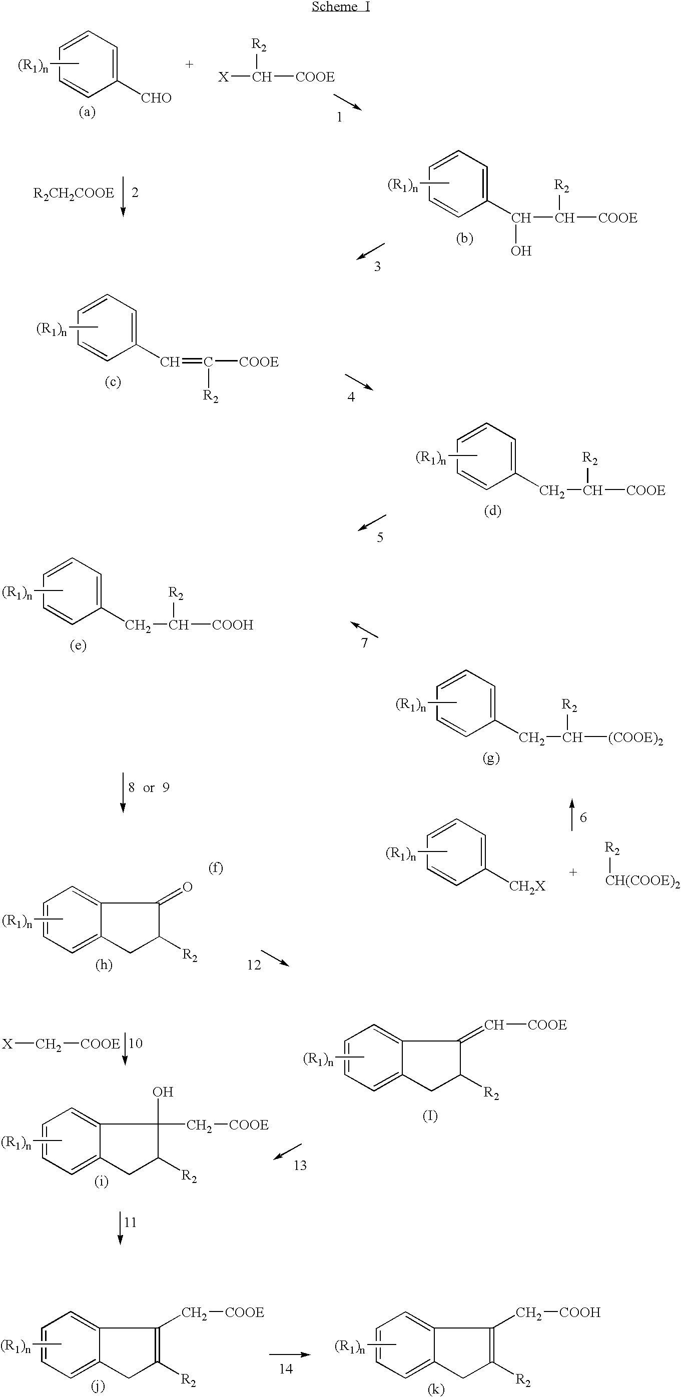

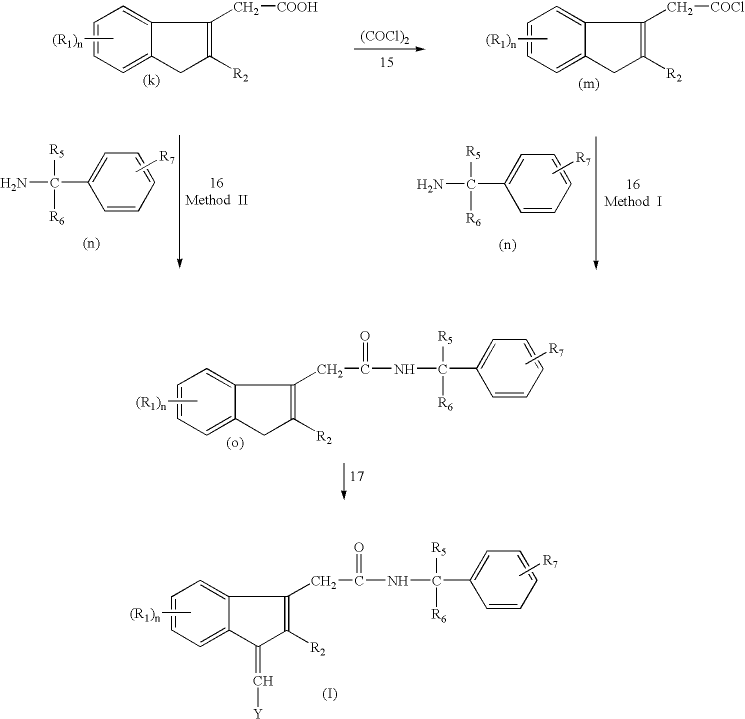

In Scheme I, several sub-variations can be used. In one sub-variation, a substituted benzaldehyde (a) may be condensed with a substituted acetic ester in a Knoevenagel reaction (see reaction 2) or with an α-halogeno propionic ester in a Reformatsky Reaction (see reactions 1 and 3). The resulting unsaturated ester (c) is hydrogenated and hydrolyzed to give a substituted benzyl propionic acid (e) (see reactions 4 and 5). Alternatively, a substituted malonic ester in a typical malonic ester synthesis (see reactions 6 and 7) and hydrolysis decarboxylation of the resulting substituted ester (g) yields the benzyl propionic acid (e) directly. This latter method is especially preferable for nitro and alkylthio substituents on the benzene ring.

The next step is the ring closure of the β-aryl proponic acid (e) to form an indanone (h) which may be carried out by a Friedel-Crafts Reaction using a Lewis acid catalyst (Cf. Organic Reactions, Vol. 2, p. 130) or by heating with polyphosphoric acid (see reactions 8 and 9, respectively). The indanone (h) may be condensed with an α-halo ester in the Reformatsky Reaction to introduce the aliphatic acid side chain by replacing the carboxyl group (see reaction 10). Alternately, this introduction can be carried out by the use of a Wittig Reaction in which the reagent is a α-triphenylphosphinyl ester, a reagent that replaces the carbonyl with a double bond to the carbon (see reaction 12). This product (1) is then immediately rearranged into the indene (j)(see reaction 13). If the Reformatsky Reaction route is used, the intermediate 3-hydroxy-3-aliphatic acid derivative i must be dehydrated to the indene (0) (see reaction 11).

The indenylacetic acid (k) in THF then is allowed to react with oxalyl or thionyl chloride or similar reagent to produce the acid chloride (m) (see reaction 15), whereupon the solvent is evaporated. There are two methods to carry out reaction 16, which is the addition of the benzylamine side chain (n).

Method ( I)

In the first method, the benzylamine (n) is added slowly at room temperature to a solution of 5-fluoro-2-methyl-3-indenylacetyl chloride in CH₂Cl₂. The reaction mixture is refluxed overnight, and extracted with aqueous HCl (10%), water, and aqueous NaHCO₃ (5%). The organic phase is dried (Na₂SO₄) and is evaporated to give the amide compound (o).

Method (II)

In the second method, the indenylacetic acid (k) in DMA is allowed to react with a carboduimide (e.g. N-(3-dimethylaminopropyl)-N′-ethylcarbodiimide hydrochloride) and benzylamine at room temperature for two days. The reaction mixture is added dropwise to stirred ice water. A yellow precipitate is filtered off, is washed with water, and is dried in vacuo. Recrystallization gives the amide compound (o).

Compounds of the type a′ (Scheme III), o (Scheme I), t (Scheme II), y (Scheme IIB) may all be used in the condensation reaction shown in Scheme III.

Substituents

X=halogen, usually Cl or Br.

E=methyl, ethyl or benzyl, or lower acyl.

R₁, R₂, R₆, R₅, and R₇=as defined in Formula I.

Y, n and m=as defined in Formula I.

Reagents and general conditions for Scheme I (numbers refer to the numbered reactions):

(1) Zn dust in anhydrous inert solvent such as benzene and ether.

(2) KHSO₄ or p-toluene sulfonic acid.

(3) NaOC₂H₅ in anhydrous ethanol at room temperature.

(4) H₂ palladium on charcoal, 40 p.s.i. room temperature.

(5) NaOH in aqueous alcohol at 20-100°.

(6) NaOC₂H₅ or any other strong base such as NaH or K-t-butoxide.

(7) Acid.

(8) Friedel-Crafts Reaction using a Lewis Acid catalyst Cf. Organic Reactions, Vol. 11, p. 130.

(9) Heat with polyphosphoric acid.

(10) Reformatsky Reaction: Zn in inert solvent, heat.

(11) p-Toluene sulfonic acid and CaCl₂ or I₂ at 200°

(12) Wittig Reaction using (C₆H₅)₃ P=C—COOE 20-80° in ether or benzene

(13) (a) NBS/CCl₄/benzoyl peroxide

(b) PtO₂/ H₂ (1 atm.)/acetic acid

(14) (a) NaOH

(b) HCl

(15) Oxalyl or thionyl chloride in CH₂Cl₂ or THF

(16) Method I: 2 equivalents of NH₂—C(R₅R₆)—Ph—(R₇)ₘ

Method II: carbodiimide in THF

(17) IN NaOCH₃ in MeOH under reflux conditions

Indanones within the scope of compound (h) in Scheme I are known in the literature and are thus readily available as intermediates for the remainder of the synthesis so that reactions 1-7 can be conveniently avoided. Among such known indanones are:

5-methoxyindanone

6-methoxyindanone

5-methylindanone

5-methyl-6-methoxyindanone

5-methyl-7-chloroindanone

4-methoxy-7-chloroindanone

4-isopropyl-2,7-dimethylindanone

5,6,7-trichloroindanone

2-n-butylindanone

5-methylthioindanone

Scheme II has two mutually exclusive sub-schemes: Scheme IIA and Scheme II B. Scheme IIA is used when R₃ is hydroxy and R₄ is hydrogen or when the two substituents form an oxo group. When R₃ is lower alkyl amino, Scheme II B is employed.

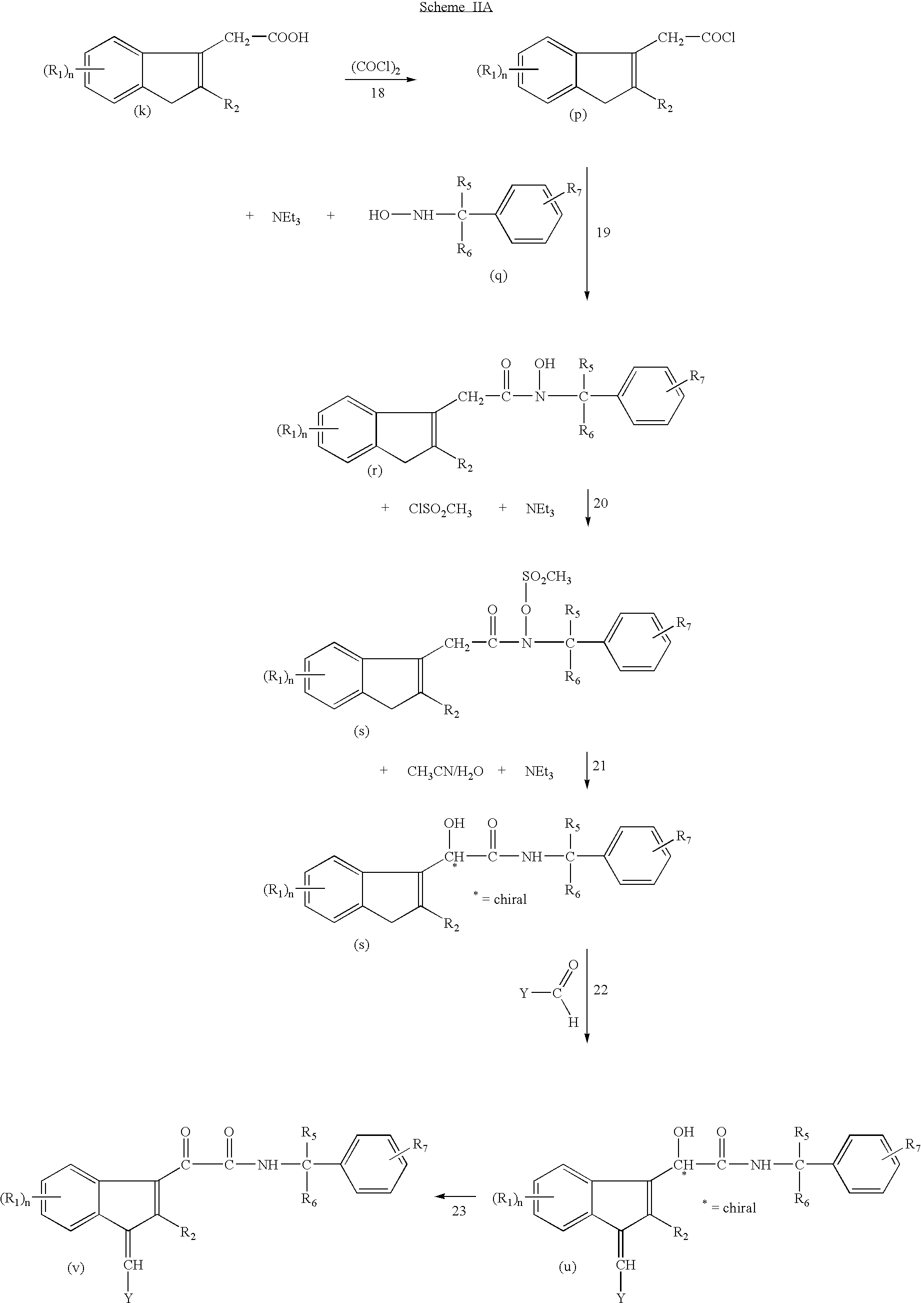

Similar to Scheme I, in Scheme IIA the indenylacetic acid (k) in THF is allowed to react with oxalylchloride under reflux conditions to produce the acid chloride (p) (see reaction 18), whereupon the solvent is evaporated. In reaction 19, a 0° C. mixture of a benzyl hydroxylamine hydrochloride (q) and Et₃N is treated with a cold solution of the acid chloride in CH₂Cl₂ over a period of 45-60 minutes. The mixture is warmed to room temperature and stirred for one hour, and is treated with water. The resulting organic layer is washed with 1 N HCl and brine, is dried over magnesium sulfate and is evaporated. The crude product, a N-hydroxy-N-benzyl acetamide (r) is purified by crystallization or flash chromatography. This general procedure is taught by Hoffman et al., JOC 1992, 57, 5700-5707.

The next step is the preparation of the N-mesyloxy amide (s) in reaction 20, which is also taught by Hoffman et al., JOC 1992, 57, 5700-5707. Specifically, to a solution of the hydroxamic acid (r) in CH₂Cl₂ at 0° C. is added triethylamine. The mixture is stirred for 10-12 minutes, and methanesulfonyl chloride is added dropwise. The mixture is stirred at 0° C. for two hours, is allowed to warm to room temperature, and is stirred for another two hours. The organic layer is washed with water, 1 N HCl, and brine, and is dried over magnesium sulfate. After rotary evaporation, the product(s) is usually purified by crystallization or flash chromatography.

The preparation of the N-benzyl-α-(hydroxy) amide (t) in reaction 21, is also taught by Hoffman et al., JOC 1992, 57, 5700-5707 and Hoffman et al., JOC 1995, 60, 4121-4125. Specifically, to a solution of the N-(mesyloxy) amide (s) in CH₃CN/H₂O is added triethylamine in CH₃CN over a period of 6-12 hours. The mixture is stirred overnight. The solvent is removed, and the residue is dissolved in ethyl acetate. The solution is washed with water, 1 N HCl, and brine, and is dried over magnesium sulfate. After rotary evaporation, the product (t) is usually purified by recrystallization.

Reaction 22 in Scheme IIA involves a condensation with certain aldehydes, which is described in Scheme III below, a scheme that is common to products made in accordance with Schemes I, IIA and IIB.

The final reaction 23 in Scheme IIA is the preparation of the N-benzyl-α-ketoamide (v), which involves the oxidation of a secondary alcohol (u) to a ketone by e.g., a Pfitzner-Moffatt oxidation, which selectively oxidizes the alcohol without oxidizing the Y group. Compounds (u) and (v) may be derivatized to obtain compounds with R₃ and R₄

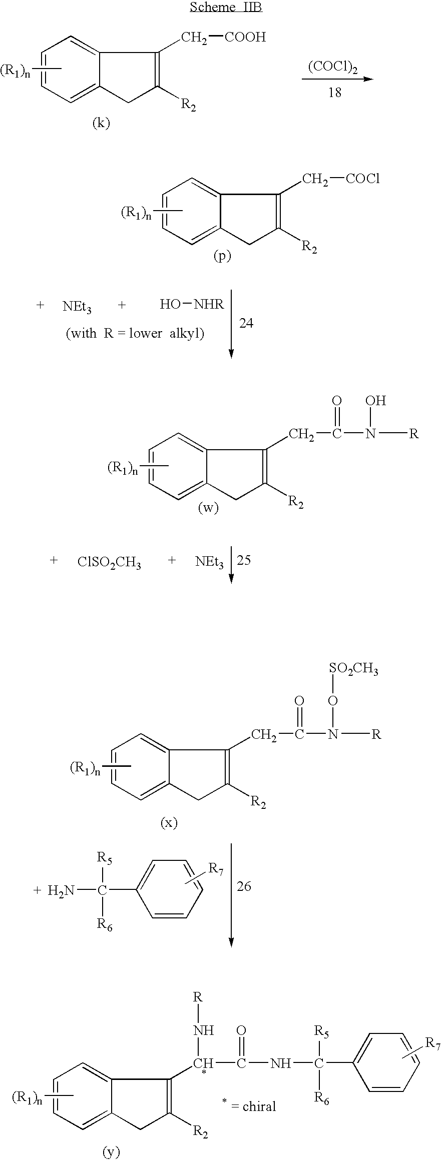

groups as set forth in Formula I.

As explained above, Scheme IIB is employed when R₃ is lower alkyl amino. Similar to Scheme I, in Scheme IIB the indenylacetic acid (k) in THF is allowed to react with oxalylchloride under reflux conditions to produce the acid chloride (p) (see reaction 18), whereupon the solvent is evaporated. In reaction 24, a mixture of an alkyl hydroxylamine hydrochloride (i.e. HO—NHR where R is a lower alkyl, preferably isopropyl) and Et₃N is treated at 0° C. with a cold solution of the acid chloride in CH₂Cl₂ over a period of 45-60 minutes. The mixture is warmed to room temperature and is stirred for one hour, and is diluted with water. The resulting organic layer is washed with 1 N HCl and brine, is dried over magnesium sulfate and is evaporated. The crude product, a N-hydroxy-N-alkyl acetamide (w) is purified by crystallization or flash chromatography. This general procedure is also taught by Hoffman et al., JOC 1992, 57, 5700-5707

The preparation of the N-mesyloxy amide (x) in reaction 25, which is also taught by Hoffman et al., JOC 1992, 57, 5700-5707. Specifically, a solution of the hydroxamic acid (w) in CH₂Cl₂ at 0° C. is treated with triethylamine, is stirred for 10-12 minutes, and is treated dropwise with methanesulfonyl chloride. The mixture is stirred at 0° C. for two hours, is allowed to warm to room temperature, and is stirred for another two hours. The resulting organic layer is washed with water, 1 N HCl, and brine, and is dried over magnesium sulfate. After rotary evaporation, the product (x) is usually purified by crystallization or flash chromatography.

The preparation of the N-benzyl indenyl-α-loweralkylamino- acetamide compound (y) in Scheme IIB as taught by Hoffman et al., JOC 1995, 60, 4121-25 and J. Am. Chem Soc. 1993, 115, 5031-34, involves the reaction of the N-mesyloxy amide (x), with a benzylamine in CH₂Cl₂ at 0° C. is added over a period of 30 minutes. The resulting solution is stirred at 0° C. for one hour and at room temperature overnight. The solvent is removed, and the residue is treated with 1 N NaOH. The extract with CH₂Cl₂ is washed with water and is dried over magnesium sulfate. After rotary evaporation, the product (y) is purified by flash chromatography or crystallization.

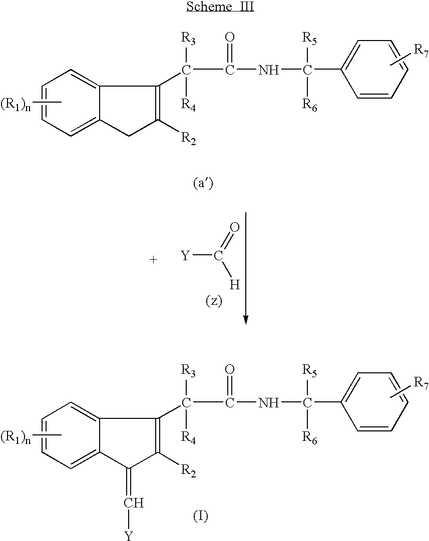

Scheme III involves the condensation of the heterocycloaldehydes (i.e., Y—CHO) with the indenyl amides to produce the final compounds of Formula I. This condensation is employed, for example, in reaction 17 in Scheme I above and in reaction 22 in Scheme IIA. It is also used to convert compound (y) in Scheme IIB to final compounds of Formula I.

In Scheme III, the amide (a′) from the above schemes, an N-heterocycloaldehyde (z), and sodium methoxide (1 M in methanol) are stirred at 60° C. under nitrogen for 24 hours. After cooling, the reaction mixture is poured into ice water. A solid is filtered off, is washed with water, and is dried in vacuo. Recrystallization provides a compound of Formula I in Schemes I and IIB and the intermediate (u) in Scheme IIA.

As has been pointed out above, it is preferable in the preparation of many types of the compounds of this invention, to use a nitro substituent on the benzene ring of the indanone nucleus and convert it later to a desired substituent since by this route a great many substituents can be reached. This is done by reduction of the nitro to the amino group followed by use of the Sandmeyer reaction to introduce chlorine, bromine, cyano or xanthate in place of the amino. From the cyano derivatives, hydrolysis yields the carboxamide and carboxylic acid; other derivatives of the carboxy group such as the esters can then be prepared. The xanthates, by hydrolysis, yield the mercapto group that may be oxidized readily to the sulfonic acid or alkylated to an alkylthio group that can then be oxidized to alkylsulfonyl groups. These reactions may be carried out either before or after the introduction of the 1-substituent.

The foregoing may be better understood from the following examples that are presented for purposes of illustration and are not intended to limit the scope of the invention. As used in the following examples, the references to substituents such as R₁, R₂, etc., refer to the corresponding compounds and substituents in Formula I above.

### EXAMPLE 1

(Z)-5-Fluoro-2-Methyl-(4-Pyridinylidene)-3-(N-Benzyl)-Indenylacetamide

(A) p-Fluoro-α-methylcinnamic acid

p-Fluorobenzaldehyde (200 g, 1.61 mol), propionic anhydride (3.5 g, 2.42 mol) and sodium propionate (155 g, 1.61 mol) are mixed in a one liter three-necked flask which had been flushed with nitrogen. The flask is heated gradually in an oil-bath to 140° C. After 20 hours, the flask is cooled to 100° C. and poured into 8 l of water. The precipitate is dissolved by adding potassium hydroxide (302 g) in 2 l of water. The aqueous solution is extracted with ether, and the ether extracts are washed with potassium hydroxide solution. The combined aqueous layers are filtered, are acidified with concentrated HCl, and are filtered. The collected solid, p-fluoro-x-methylcinnamic acid, is washed with water, and is dried and used as obtained.

(B) p-Fluoro-α-methylhydrocinnamic acid

To p-fluoro-α-methylcinnamic acid (177.9 g, 0.987 mol) in 3.6 l ethanol is added 11.0 g of 5% Pd/C. The mixture is reduced at room temperature under a hydrogen pressure of 40 p.s.i. When hydrogen uptake ceases, the catalyst is filtered off, and the solvent is evaporated in vacuo to give the product, p-fluoro-α-methylhydrocinnamic acid, which was used directly in the next step.

(C) 6-Fluoro-2-methylindanone

To 932 g polyphosphoric acid at 70° C. (steam bath) is added p-fluoro-α-methylhydrocinnamic acid (93.2 g, 0.5 mol) slowly with stirring. The temperature is gradually raised to 95° C., and the mixture is kept at this temperature for 1 hour. The mixture is allowed to cool and is added to 2 l. of water. The aqueous suspension is extracted with ether. The extract is washed twice with saturated sodium chloride solution, 5% Na₂CO₃ solution, and water, and is dried, and is concentrated on 200 g silica-gel; the slurry is added to a five pound silica-gel column packed with 5% ether-petroleum ether. The column is eluted with 5-10% ether-petroleum ether, to give 6-fluoro-2-methylindanone. Elution is followed by TLC.

(D) 5-fluoro-2-methylindenyl-3-acetic acid

A mixture of 6-fluoro-2-methylindanone (18.4 g, 0.112 mol), cyanoacetic acid (10.5 g, 0.123 mol), acetic acid (6.6 g), and ammonium acetate (1.7 g) in dry toluene (15.5 ml) is refluxed with stirring for 21 hours, as the liberated water is collected in a Dean Stark trap. The toluene is evaporated, and the residue is dissolved in 60 ml of hot ethanol and 14 ml of 2.2 N aqueous potassium hydroxide solution. 22 g of 85% KOH in 150 ml of water is added, and the mixture refluxed for 13 hours under nitrogen. The ethanol is removed under vacuum, and 500 ml water is added. The aqueous solution is extracted well with ether, and is then boiled with charcoal. The aqueous filtrate is acidified to pH 2 with 50% cold hydrochloric acid. The precipitate is dried and 5-fluoro-2-methylindenyl-3-acetic acid (M.P. 164-166° C.) is obtained.

(E) 5-fluoro-2-methylindenyl-3-acetyl chloride

5-fluoro-2-methylindenyl-3-acetic acid (70 mmol) in THF (70 ml) is allowed to react with oxalylchloride (2 M in CH₂Cl₂; 35 ml; 70 mmol) under reflux conditions (24 hours). The solvent is evaporated to yield the title compound, which is used as such in the next step.

(F) 5-Fluoro-2-methyl-3-(N-benzyl)-indenylacetamide

Benzylamine (5 mmol) is added slowly at room temperature to a solution of 5-fluoro-2-methylindenyl-3-acetyl chloride (2.5 mmol.) in CH₂Cl₂ (10 ml). The reaction mixture is refluxed overnight, and is extracted with aqueous HCl (10%), water, and aqueous NaHCO₃ (5%). The organic phase is dried (Na₂SO₄) and is evaporated to give the title compound, which is recrystallized from CH₂Cl₂ to give the title compound as a white solid (m.p. 144° C.).

(G) (Z)-5-Fluoro-2-methyl-(4-pyridinylidene)-3-(N-benzyl)-indenylacetamide

5-fluoro-2-methyl-3-(N-benzyl)-indenylacetamide (3.38 mmol), 4-pyridinecarboxaldehyde (4 mmol), sodium methoxide (1M NaOCH₃ in methanol (30 ml)) are heated at 60° C. under nitrogen with stirring for 24 hours. After cooling, the reaction mixture is poured into ice water (200 ml). A solid is filtered off, washed with water, and dried in vacuo. Recrystallization from CH₃CN gives the title compound (m.p. 202° C.) as a yellow solid (R₁═F, R₂═CH₃, R₃═H, R₄═H, R₅═H, R₆═H, R₇═H, n=1, m=1, Y=4-pyridinyl).

(H) (E)-5-Fluoro-2-methyl-(4-pyridinylidene)-3-(N-benzyl)-indenylacetamide

The mother liquor obtained from the CH₃CN recrystallization of 1G is rich on the geometrical isomer of 1G. The E-isomer can be obtained pure by repeated recrystallizations from CH₃CN.

### EXAMPLE 2

(Z)-5-Fluoro-2-Methyl-(3-Pyridinylidene)-3-(N-Benzyl)-Indenyl acetamide

This compound is obtained from 5-fluoro-2-methyl-3-(N-benzyl)-indenylacetamide (Example 1F) using the procedure of Example 1, part G and replacing 4-pyridinecarboxaldehyde with 3-pyridinecarboxaldehyde. Recrystallization from CH₃CN gives the title compound (m.p. 175° C.)(R₁═F, R₂═CH₃, R₃═H, R₄═H, R₅═H, R₆═H, R₇═H, n=1, m=1, Y=3-pyridinyl).

### EXAMPLE 3

(Z)-5-Fluoro-2-Methyl-(2-Pyridinylidene)-3-(N-Benzyl)-Indenylacetamide

This compound is obtained from 5-fluoro-2-methyl-3-(N-benzyl)-indenylacetamide (Example 1F) using the procedure of Example 1, part G and replacing 4-pyridinecarboxaldehyde with 2-pyridinecarboxaldehyde. Recrystallization from ethylacetate gives the title compound (m.p. 218° C.)(R₁═F, R₂═CH₃, R₃═H, R₄═H, R₅═H, R₆═H, R₇═H, n=1, m=1, Y=2-pyridinyl).

### EXAMPLE 4

(Z)-5-Fluoro-2-Methyl-(4-Quinolinylidene)-3-(N-Benzyl)-Indenylacetamide

This compound is obtained from 5-fluoro-2-methyl-3-(N-benzyl)-indenylacetamide (Example 1F) using the procedure of Example 1, part G and replacing 4-pyridinecarboxaldehyde with 4-quinolinecarboxaldehyde. Recrystallization from ethylacetate gives the title compound (m.p. 239° C.)(R₁═F, R₂═CH₃, R₃═H, R₄═H, R₅═H, R₆═H, R₇═H, n=1, m=1, Y=4-quinolinyl).

### EXAMPLE 5

(Z)-5-Fluoro-2-Methyl-(4,6-Dimethyl-2-Pyridinylidene)-3-(N-Benzyl)-Indenylacetamide

5-Fluoro-2-methyl-3-(N-benzyl)-indenylacetamide from Example 1, part F is allowed to react with 4,6-dimethyl-2-pyridinecarboxaldehyde according to the procedure of Example, 1, part G in order to obtain the title compound. Recrystallization gives the title compound (R₁═F, R₂═CH₃, R₃═H, R₄═H, R₅═H, R₆═H, R₇═H, n=1, m=1, Y=4,6-dimethyl-2-pyridinyl).

### EXAMPLE 6

(Z)-5-Fluoro-2-Methyl-(3-Quinolinylidene)-3-(N-Benzyl)-Indenylacetamide

5-Fluoro-2-methyl-3-(N-benzyl)-indenylacetamide from Example 1, part F is allowed to react with 3-quinolinecarboxaldehyde according to the procedure of Example 1, part G in order to obtain the title compound. Recrystallization gives the title compound (R₁═F, R₂═CH₃, R₃═H, R₄═H, R₅═H, R₆═H, R₇═H, n=1, m=1, Y=3-quinolinyl)

### EXAMPLE 7

(Z)-5-Fluoro-2-Methyl-(2-Quinolinylidene)-3-(N-Benzyl)-Indenylacetamide

5-Fluoro-2-methyl-3-(N-benzyl)-indenylacetamide from Example 1, part F is allowed to react with 2-quinolinecarboxaldehyde according to the procedure of Example 1, part G in order to obtain the title compound. Recrystallization gives the title compound (R₁═F, R₂═CH₃, R₃═H, R₄═H, R₅═H, R₆═H, R₇═H, n=1, m=1, Y=2-quinolinyl).

### EXAMPLE 8

(Z)-5-Fluoro-2-Methyl-(Pyrazinylidene)-3-(N-Benzyl)-Indenylacetamide

5-Fluoro-2-methyl-3-(N-benzyl)-indenylacetamide from Example 1, part F is allowed to react with pyrazinealdehyde according to the procedure of Example 1, part G in order to obtain the title compound. Recrystallization gives the title compound (R₁═F, R₂═CH₃, R₃═H, R₄═H, R₅═H, R₆═H, R₇═H, n=1, m=1, Y=pyrazinyl).

### EXAMPLE 9

(Z)-5-Fluoro-2-Methyl-(3 -Pyridazinylidene)-3-(N-Benzyl)-Indenylacetamide

5-Fluoro-2-methyl-3-(N-benzyl)-indenylacetamide from Example 1, part F is allowed to react with pyridazine-3-aldehyde according to the procedure of Example 1, part G in order to obtain the title compound. Recrystallization gives the title compound (R₁═F, R₂═CH₃, R₃═H, R₄═H, R₅═H, R₆═H, R₇═H, n=1, m=1, Y=3-pyridazinyl).

### EXAMPLE 10

(Z)-5-Fluoro-2-Methyl-(4-Pyrimidinylidene)-3-(N-Benzyl)-Indenylacetamide

5-Fluoro-2-methyl-3-(N-benzyl)-indenylacetamide from Example 1, part F is allowed to react with pyrimidine-4-aldehyde according to the procedure of Example 1, part G in order to obtain the title compound. Recrystallization gives the title compound (R₁═F, R₂═CH₃, R₃═H, R₄═H, R₅═H, R₆═H, R₇═H, n=1, m=1, Y=4-pyrimidinyl).

### EXAMPLE 11

(Z)-5-Fluoro-2-Methyl-(2-Methyl-4-Pyrimidinylidene)-3-(N-Benzyl)-Indenylacetamide

5-Fluoro-2-methyl-3-(N-benzyl)-indenylacetamide from Example 1, part F is allowed to react with 2-methyl-pyrimidine-4-aldehyde according to the procedure of Example 1, part G in order to obtain the title compound. Recrystallization gives the title compound (R₁═F, R₂═CH₃, R₃═H, R₄═H, R₅═H, R₆═H, R₇═H, n=1, m=1, Y=2-methyl-4-pyrimidinyl).

### EXAMPLE 12

(Z)-5-Fluoro-2-Methyl-(4-Pyridazinylidene)-3-(N-Benzyl)-Indenylacetamide

5-Fluoro-2-methyl-3-(N-benzyl)-indenylacetamide from Example 1, part F is allowed to react with pyridazine-4-aldehyde according to the procedure of Example 1, part G in order to obtain the title compound. Recrystallization gives the title compound (R₁═F, R₂═CH₃, R₃═H, R₄═H, R₅═H, R₆═H, R₇═H, n=1, m=1, Y=4-pyridazinyl).

### EXAMPLE 13

(Z)-5-Fluoro-2-Methyl-(1-Methyl-3-Indolylidene)-3-(N-Benzyl)-Indenylacetamide

5-Fluoro-2-methyl-3-(N-benzyl)-indenylacetamide from Example 1, part F is allowed to react with 1-methylindole-3-carboxaldehyde according to the procedure of Example 1, part G in order to obtain the title compound. Recrystallization gives the title compound (R₁═F, R₂═CH₃, R₃═H, R₄═H, R₅═H, R₆═H, R₇═H, n=1, m=1, Y=1-methyl-3-indolyl).

### EXAMPLE 14

(Z)-5-Fluoro-2-Methyl-( 1 -Acetyl-3-Indolylidene)-3-(N-Benzyl)-Indenylacetamide

5-Fluoro-2-methyl-3-(N-benzyl)-indenylacetamide from Example 1, part F is allowed to react with 1-acetyl-3-indolecarboxaldehyde according to the procedure of Example 1, part G in order to obtain the title compound. Recrystallization gives the title compound (R₁═F, R₂═CH₃, R₃═H, R₄═H, R₅═H, R₆═H, R₇═H, n=1, m=1, Y=1-acetyl-3-indolyl).

### EXAMPLE 15

(Z)-5-Fluoro-2-Methyl-(4-Pyridinylidene)-3-(N-2-Fluorobenzyl)-Indenylacetamide

(A) 5-Fluoro-2-methyl-3-(N-2-fluorobenzyl)-indenylacetamide

This compound is obtained from 5-fluoro-2-methylindenyl-3-acetyl chloride (Example 1E) using the procedure of Example 1, Part F and replacing benzylamine with 2-fluorobenzylamine.

(B) (Z)-5-Fluoro-2-methyl-(4-pyridinylidene)-3-(N-2-fluorobenzyl)-indenylacetamide

5-Fluoro-2-methyl-3-(N-2-fluorobenzyl)-indenylacetamide is allowed to react with 4-pryidinecarboxaldehyde according to the procedure of Example 1, part G in order to obtain the title compound. Recrystallization gives the title compound (R₁═F, R₂═CH₃, R₃═H, R₄═H, R₅═H, R₆═H, R₇═F, n=1, m=1, Y=4-pyridinyl).

### EXAMPLE 16

(Z)-5-Fluoro-2-Methyl-(3-Pyridinylidene)-3-(N-2-Fluorobenzyl)-Indenylacetamide

5-Fluoro-2-methyl-3-(N-2-fluorobenzyl)-indenylacetamide from Example 15, part A is allowed to react with 3-pryidinecarboxaldehyde according to the procedure of Example 1, part G in order to obtain the title compound. Recrystallization gives the title compound (R₁═F, R₂═CH₃, R₃═H, R₄═H, R₅═H, R₆═H, R₇═F, n=1, m=1, Y=3-pyridinyl).

### EXAMPLE 17

(Z)-5-Fluoro-2-Methyl-(2-Pyridinylidene)-3-(N-2-Fluorobenzyl)-Indenylacetamide

5-Fluoro-2-methyl-3-(N-2-fluorobenzyl)-indenylacetamide from Example 15, part A is allowed to react with 2-pyridinecarboxaldehyde according to the procedure of Example 1, part G in order to obtain the title compound. Recrystallization gives the title compound (R₁═F, R₂═CH₃, R₃═H, R₄═H, R₅═H, R₆═H, R₇═F, n=1, m=1, Y=2-pyridinyl).

### EXAMPLE 18

(Z)-5-Fluoro-2-Methyl-(4-Quinolinylidene)-3-(N-2-Fluorobenzyl)-Indenylacetamide

5-Fluoro-2-methyl-3-(N-2-fluorobenzyl)-indenylacetamide from Example 15, part A is allowed to react with 4-quinolinecarboxaldehyde according to the procedure of Example 1, part G in order to obtain the title compound. Recrystallization gives the title compound (R₁═F, R₂═CH₃, R₃═H, R₄═H, R₅═H, R₆═H, R₇═F, n=1, m=1, Y=3-quinolinyl).

### EXAMPLE 19

(Z)-5-Fluoro-2-Methyl-(3-Pyrazinylidene)-3-(N-2-Fluorobenzyl)-Indenylacetamide

5-Fluoro-2-methyl-3-(N-2-fluorobenzyl)-indenylacetamide from Example 15, part A is allowed to react with pyrazinealdehyde according to the procedure of Example 1, Part G in order to obtain the title compound. Recrystallization gives the title compound (R₁═F, R₂═CH₃, R₃═H, R₄═H, R₅═H, R₆═H, R₇═F, n=1, m=1, Y=3-pyrazinyl).

### EXAMPLE 20

(Z)-5-Fluoro-2-Methyl-(3-Pyridazinylidene)-3-(N-2-Fluorobenzyl)-Indenylacetamide

5-Fluoro-2-methyl-3-(N-2-fluorobenzyl)-indenylacetamide from Example 15, part A is allowed to react with 3-pyridazine-3-aldehyde according to the procedure of Example 1, Part G in order to obtain the title compound. Recrystallization gives the title compound(R₁═F, R₂═CH₃, R₃═H, R₄═H, R₅═H, R₆═H, R₇═F, n=1, m=1, Y=3-pyridazinyl).

### EXAMPLE 21

(Z)-5-Fluoro-2-Methyl-(3 -Pyrimidinylidene)-3-(N-2-Fluorobenzyl)-Indenylacetamide

5-Fluoro-2-methyl-3-(N-2-fluorobenzyl)-indenylacetamide from Example 15, part A is allowed to react with pryimidine-4-aldehyde according to the procedure of Example 1, Part G in order to obtain the title compound. Recrystallization gives the title compound (R₁═F, R₂═CH₃, R₃═H, R₄═H, R₅═H, R₆═H, R₇═F, n=1, m=1, Y=3-pyrimidinyl).

### EXAMPLE 22

(Z)-5-Fluoro-2-Methyl-(4-Pyridazinylidene)-3-(N-2-Fluorobenzyl)-Indenylacetamide

5-Fluoro-2-methyl-3-(N-2-fluorobenzyl)-indenylacetamide from Example 15, part A is allowed to react with pryidazine-4-aldehyde according to the procedure of Example 1, Part G in order to obtain the title compound. Recrystallization gives the title compound (R₁═F, R₂═CH₃, R₃═H, R₄═H, R₅═H, R₆═H, R₇═F, n=1, m=1, Y=4-pyridazinyl).

### EXAMPLE 23

(Z)-5-Fluoro-2-Methyl-(4-Pyridinylidene)-3-(N-(S-α-Hydroxymethyl)Benzyl)-Indenylacetamide

(A) 5-Fluoro-2-methyl-3-(N-(S-α-hydroxylmethyl)benzyl)-indenylacetamide

5-Fluoro-2-methylindenyl-3-acetic acid (from Example 1D) (2.6 mmol) in DMA (2 ml) is allowed to react with n-(3-dimethylaminopropyl)-N′-ethylcarbodiimide hydrochloride (4 mmol) and S-2-amino-2-phenylethanol (3.5 mmol) at room temperature for two days. The reaction mixture is added dropwise to stirred ice water (50 ml). A white precipitate is filtered off, washed with water (5 ml), and dried in vacuo. Recrystallization from ethylacetate gives the desired compound.

(B) (Z)-5-fluoro-2-methyl-(4-pyridinylidene)-3-(N-(S-α-hydroxymethyl)benzyl)-indenylacetamide

5-Fluoro-2-methyl-3-(N-(S-α-hydroxylmethyl)benzyl)-indenylacetamide from part A is allowed to react with 4-pryidinecarboxaldehyde according to the procedure of Example 1, Part G in order to obtain the title compound. Recrystallization gives the title compound (R₁═F, R₂═CH₃, R₃═H, R₄═H, R₅═CH₂OH, R₆═H, R₇═H, n=1, m=1, Y=4-pyridinyl).

### EXAMPLE 24

(Z)-5-Fluoro-2-Methyl-(3-Pyridinylidene)-3-(N-(S-α-Hydroxymethyl)Benzyl)-Indenylacetamide

5-Fluoro-2-methyl-3-(N-(S-α-hydroxylmethyl)benzyl)-indenylacetamide from Example 23 part A is allowed to react with 3-pryidinecarboxaldehyde according to the procedure of Example 1, Part G in order to obtain the title compound. Recrystallization gives the title compound (R₁═F, R₂═CH₃, R₃═H, R₄═H, R₅═CH₂OH, R₆═H, R₇═H, n=1, m=1, Y=3-pyridinyl).

### EXAMPLE 25

(Z)-5-Fluoro-2-Methyl-(2-Pyridinylidene)-3-(N-(S-α-Hydroxymethyl)Benzyl)-Indenylacetamide

5-Fluoro-2-methyl-3-(N-(S-α-hydroxylmethyl)benzyl)-indenylacetamide from Example 23 part A is allowed to react with 2-pryidinecarboxaldehyde according to the procedure of Example 1, Part G in order to obtain the title compound. Recrystallization gives the title compound (R₁═F, R₂═CH₃, R₃═H, R₄═H, R₅═CH₂OH, R₆═H, n=1, m=1, Y=2-pyridinyl).

### EXAMPLE 26

(Z)-5-Fluoro-2-Methyl-(4-Quinolinylidene)-3-(N-(S-α-Hydroxymethyl)Benzyl)-Indenylacetamide

5-Fluoro-2-methyl-3-(N-(S-α-hydroxylmethyl)benzyl)-indenylacetamide from Example 23 part A is allowed to react with 4-quinolinecarboxaldehyde according to the procedure of Example 1, Part G in order to obtain the title compound. Recrystallization gives the title compound (R₁═F, R₂═CH₃, R₃═H, R₄═H, R₅═CH₂OH, R₆═H, R₇═H, n=1, m=1, Y=4-quinolinyl).

### EXAMPLE 27

(Z)-5-Fluoro-2-Methyl-(Pyrazidinylidene)-3-(N-(S-α-Hydroxymethyl)Benzyl) Indenylacetamide

5-Fluoro-2-methyl-3-(N-(S-α-hydroxylmethyl)benzyl)-indenylacetamide from Example 23 part A is allowed to react with pryazidinecarboxaldehyde according to the procedure of Example 1, Part G in order to obtain the title compound. Recrystallization gives the title compound (R₁═F, R₂═CH₃, R₃═H, R₄═H, R₅═CH₂OH, R₆═H, n=1, m=1, Y=pyrazidinyl).

### EXAMPLE 28

(Z)-5-Fluoro-2-Methyl-(3-Pyridazinylidene)-3-(N-(S-α-Hydroxymethyl)Benzyl)-Indenylacetamide

5-Fluoro-2-methyl-3-(N-(S-α-hydroxylmethyl)benzyl)-indenylacetamide from Example 23 part A is allowed to react with pryidazine-3-aldehyde according to the procedure of Example 1, Part G in order to obtain the title compound. Recrystallization gives the title compound (R₁═F, R₂═CH₃, R₃═H, R₄═H, R₅═CH₂OH, R₆═H, n=1, m=1, Y=3-pyridazinyl).

### EXAMPLE 29

(Z)-5-Fluoro-2-Methyl-(4-Pyrimidinylidene)-3-(N-(S-α-Hydroxymethyl)Benzyl)-Indenylacetamide

5-Fluoro-2-methyl-3-(N-(S-α-hydroxylmethyl)benzyl)-indenylacetamide from Example 23 part A is allowed to react with pryimidine-4-aldehyde according to the procedure of Example 1, Part G in order to obtain the title compound. Recrystallization gives the title compound (R₁═F, R₂═CH₃, R₃═H, R₄═H, R₅═CH₂OH, R₆═H, n=1, m=1, Y=4-pyrimidinyl).

### EXAMPLE 30

(Z)-5-Fluoro-2-Methyl-(4-Pyridazinylidene)-3-(N-(S-α-Hydroxymethyl)Benzyl)-Indenylacetamide

5-Fluoro-2-methyl-3-(N-(S-α-hydroxylmethyl)benzyl)-indenylacetamide from Example 23 part A is allowed to react with pryidazine-4-aldehyde according to the procedure of Example 1, Part G in order to obtain the title compound. Recrystallization gives the title compound (R₁═F, R₂═CH₃, R₃═H, R₄═H, R₅═CH₂OH, R₆═H, n=1, m=1, Y=4-pyridazinyl).

### EXAMPLE 31

rac-(Z)-5-Fluoro-2-Methyl-(4-Pyridinylidene)-3-(N-Benzyl)Indenyl-α-Hydroxyacetamide

(A) 5-fluoro-2-methyl-3-(N-benzyl-N-hydroxy)-indenylacetamide

To a mixture of N-benzylhydroxylamine hydrochloride (12 mmol) and Et₃N (22 mmol) in CH₂Cl₂ (100 ml) at 0° C. is added a cold solution of 5-fluoro-2-methylindenyl-3-acetyl chloride (Example 1, Step E) (10 mmol) in CH₂Cl₂ (75 ml) over a period of 45-60 minutes. The mixture is warmed to room temperature and is stirred for 1 hour. The mixture is diluted with water (100 ml), and the organic layer is washed with HCl (2×25 ml) and brine (2×100 ml), dried (MgSO₄) and evaporated. The crude product is purified with flash chromatography to give the title compound.

(B) 5-Fluoro-2-methyl-3-(N-benzyl-N-mesyloxy)-indenylacetamide

To a solution of 5-fluoro-2-methyl-3-(N-benzyl-N-hydroxy)-indenylacetamide (5 mmol) in CH₂Cl₂ (25 ml) at 0° C. is added triethylamine (5 mmol). The mixture is stirred for 10 minutes, and methanesulfonyl chloride (5.5 mmol) is added dropwise. The solution is stirred at 0° C. for 2 hours, allowed to warm to room temperature, and stirred for another 2 hours. The organic layer is washed with water (2×20 ml), in HCl (15 ml), and brine (20 ml) and dried over MgSO₄. After rotary evaporation, the product is purified with flash chromatography to give the title compound.

(C) rac-5-Fluoro-2-methyl-3-(N-benzyl)-α-hydroxyindenylacetamide

To a solution of 5-fluoro-2-methyl-3-(N-benzyl-N-mesyloxy)-indenylacetamide (2 mmol) in CH₃CN/H₂O (12 ml. each) is added triethylamine (2.1 mmol) in CH₃CN (24 ml) over a period of 6 hours. The mixture is stirred overnight. The solvent is removed, and the residue diluted with ethyl acetate (60 ml), washed with water (4×20 ml), in HCl (15 ml), and brine (20 ml) and dried over MgSO₄. After rotary evaporation, the product is purified by recrystallization to give the title compound.

(D) rac-(Z)-5-Fluoro-2-methyl-(4-pyridinylidene)-3-(N-benzyl)-indenyl-α-hydroxyacetamide is obtained from rac-5-fluoro-2-methyl-3-(N-benzyl)-α-hydroxyindenylacetamide using the procedure of Example 1, Part G (R₁═F, R₂═CH₃, R₃═OH, R₄═H, R₅═H, R₆═H, R₇═H, n=1, m=1, Y=4-pyridinyl).

### EXAMPLE 32

2-[(Z)-5-Fluoro-2-Methyl-(4-Pyridinylidene)-3-(N-Benzyl)-Indenyl]-Oxyacetamide

For Pfitzner-Moffatt oxidation, a solution of rac-(Z)-5-fluoro-2-methyl-(4-pyridinylidene)-3-(N-benzyl)-indenyl-α-hydroxyacetamide (1 mmol) in DMSO (5 ml) is treated with dicyclohexylcarbodiimide (3 mmol). The mixture is stirred overnight, and the solvent is evaporated. The crude product is purified by flash chromatography to give the title compound (R₁═F, R₂═CH₃, R₃ and R₄ together form C═O, R₅═H, R₆═H, R₇═H, n=1, m=1, and Y=4-pyridinyl).

### EXAMPLE 33

rac-(Z)-5-Fluoro-2-Methyl-(4-Pyridinylidene)-3-(N-Benzyl)-Indenyl-α-(2-Propylamino)-Acetamide

(A) 5-Fluoro-2-methyl-3-(N-2-propyl-N-hydroxy)-indenylacetamide is obtained from 5-fluoro-2-methylindenyl-3-acetyl chloride (Example 1, Step E) using the procedure of Example 31, Part A and replacing N-benzylhydroxylamine hydrochloride with N-2-propyl hydroxylamine hydrochloride.

(B) 5-Fluoro-2-methyl-3-(N-2-propyl-N-mesyloxy)-indenylacetamide is obtained according to the procedure of Example 31, Part B.

(C) rac-5-Fluoro-2-methyl-3-(N-benzyl)-α-(2-propylamino)-acetamide. To 5-fluoro-2-methyl-3-(N-2-propyl-N-mesyloxy)-indenylacetamide (2 mmol) in CH₂Cl₂ (25 ml) at 0° C. is added benzylamine (4.4 mmol) in CH₂Cl₂ (15 ml) over a period of 30 minutes. The resulting solution is stirred at 0° C. for 1 hour, and at room temperature overnight. The solvent is removed, and the residue is treated with 1 N NaOH, and is extracted with CH₂Cl₂ (100 ml). The extract is washed with water (2×10 ml), and is dried over MgSO₄. After rotary evaporation, the product is purified by flash chromatography.

(D) rac-(Z)-5-Fluoro-2-methyl-(4-pyridinylidene)-3-(N-benzyl)-indenyl-α-(2-propylamino)-acetamide is obtained from rac-5-fluoro-2-methyl-3-(N-benzyl)-α-(2-propylamino)-acetamide using the procedure of Example 1, Part G (R₁═F, R₂═CH₃, R₃=isopropylamino, R₄═H, R₅═H, R₆═H, R₇═H, n=1, m=1, Y=4-pyridinyl).

### EXAMPLE 34

(Z)-6-Methoxy-2-Methyl-(4-Pyridinylidene)-3 -(N-Benzyl)-Indenylacetamide

(A) Ethyl-2-Hydroxy-2-(p-Methoxyphenol)-1-Methylpropionate

In a 500 ml. 3-necked flask is placed 36.2 g. (0.55 mole) of zinc dust, a 250 ml. addition funnel is charged with a solution of 80 ml. anhydrous benzene, 20 ml. of anhydrous ether, 80 g. (0.58 mole) of p-anisaldehyde and 98 g. (0.55 mole) of ethyl-2-bromoproplonate. About 10 ml. of the solution is added to the zinc dust with vigorous stirring, and the mixture is warmed gently until an exothermic reaction commences. The remainder is added dropwise at such a rate that the reaction mixture continues to reflux smoothly (ca. 30-35 min.). After addition is completed the mixture is placed in a water bath and refluxed for 30 minutes. After cooling to 0°, 250 ml. of 10% sulfuric acid is added with vigorous stirring. The benzene layer is extracted twice with 50 ml. portions of 5% sulfuric acid and washed twice with 50 ml. portions of water. The combined aqueous acidic layers are extracted with 2×50 ml. ether. The combined etheral and benzene extracts are dried over sodium sulfate. Evaporation of solvent and fractionation of the residue through a 6″ Vigreux column affords 89 g. (60%) of the product, ethyl-2-hydroxy-2-(p-methoxyphenyl)-1-methylpropionate, B.P. 165-160° (1.5 mm.).

(B) 6-Methoxy-2-methylindanone

By the method described in Vander Zanden, Rec. Trav. Chim., 68, 413 (1949), the compound from part A is converted to 6-methoxy-2-methylindanone.

Alternatively, the same compound can be obtained by adding α-methyl-β-(p-methoxylphenyl)propionic acid (15 g.) to 170 g. of polyphosphoric acid at 50° and heating the mixture at 83-90° for two hours. The syrup is poured into iced water. The mixture is stirred for one-half hour, and is extracted with ether (3×). The etheral solution is washed with water (2×) and 5% NaHCO₃ (5×) until all acidic material has been removed, and is dried over sodium sulfate. Evaporation of the solution gives 9.1 g. of the indanone as a pale yellow oil.

(C) (Z)-6-Methoxy-2-methyl-(4-pyridinylidene)-3-(N-benzyl)-indenylacetamide

In accordance with the procedures described in Example 1, parts D-G, this compound is obtained substituting 6-methoxy-2-methylindanone for 6-fluoro-2-methylindanone in part D of Example 1.

### EXAMPLE 35

(Z)-5-Methoxy-2-Methyl-(4-Pyridinylidene)-3-(N-Benzyl)-Indenylacetamide

(A) Ethyl 5-Methoxy-2-Methyl-3-Indenyl Acetate

A solution of 13.4 g of 6-methoxy-2-methylindanone and 21 g. of ethyl bromoacetate in 45 ml. benzene is added over a period of five minutes to 21 g. of zinc amalgam (prepared according to Org. Syn. Coll. Vol. 3) in 110 ml. benzene and 40 ml. dry ether. A few crystals of iodine are added to start the reaction, and the reaction mixture is maintained at reflux temperature (ca. 65°) with external heating. At three-hour intervals, two batches of 10 g. zinc amalgam and 10 g. bromoester are added and the mixture is then refluxed for 8 hours. After addition of 30 ml. of ethanol and 150 ml. of acetic acid, the mixture is poured into 700 ml. of 50% aqueous acetic acid. The organic layer is separated, and the aqueous layer is extracted twice with ether. The combined organic layers are washed thoroughly with water, ammonium hydroxide and water. Drying over sodium sulfate, evaporation of solvent in vacuo followed by pumping at 80° (bath temperature)(1-2 mm.) gives crude ethyl-(1 -hydroxy-2-methyl-6-methoxy-indenyl) acetate (ca. 18 g.).

A mixture of the above crude hydroxyester, 20 g. of p-toluenesulfonic acid monohydrate and 20 g. of anhydrous calcium chloride in 250 ml. toluene is refluxed overnight. The solution is filtered, and the solid residue is washed with toluene. The combined toluene solution is washed with water, sodium bicarbonate, water and then dried over sodium sulfate. After evaporation, the crude ethyl 5-methoxy-2-methyl-3-indenyl acetate is chromatographed on acid-washed alumina, and the product is eluted with petroleum ether-ether (v./v. 50-100%) as a yellow oil (11.8 g., 70%).

(B) (Z)-5-Methoxy-2-methyl-(4-pyridinylidene)-3-(N-benzyl)-indenylacetamide

In accordance with the procedures described in Example 1, parts E-G, this compound is obtained substituting ethyl-5-methoxy-2-methyl-3-indenyl acetate for 5-fluoro-2-methindenyl-3-acetic acid in Example 1, part E.

### EXAMPLE 36

(Z)-α-5-Methoxy-2-Methyl-(4-Pyridinylidene)-3-(N-Benzyl)-Indenylpropionamide

(A) α-(5 -Methoxy-2-methyl-3-indenyl)propionic acid

The procedure of Example 35, part (A) is followed using ethyl α-bromopropionate in equivalent quantities in place of ethyl bromoacetate used therein. There is obtained ethyl α-(1-hydroxy-6-methoxy-2-methyl-1-indanyl)propionate, which is dehydrated to ethyl α-(5-methoxy-2-methyl-3-indenyl)propionate in the same manner.

The above ester is saponified to give α-(5-methoxy-2-methyl-3-indenyl)propionic acid.

(B) (Z)-α-5-Methoxy-2-methyl-(4-pyridinyl)-3-(N-benzyl)-α-methyl indenylpropionamide

In accordance with the procedures described in Example 1, parts E-G, this compound is obtained substituting α-5-methoxy-2-methyl-3-indenyl)propionic acid for 5-fluoro-2-methylindenyl-3-acetic acid in Example 1, part E.

### EXAMPLE 37

(Z) α-Fluoro-5-Methoxy-2-Methyl-(4-Pyridinylidene)-3-(N-Benzyl)Indenylacetamide

(A) Methyl-5-Methoxy-2-Methyl-3-Indenyl-α-Fluoro Acetate

A mixture of potassium fluoride (0.1 mole) and methyl-5-methoxy-2-methyl-3-indenyl-α-tosyloxy acetate (0.05 mole) in 200 ml. dimethylformamide is heated under nitrogen at the reflux temperature for 2-4 hours. The reaction mixture is cooled, poured into iced water and then extracted with ether. The ethereal solution is washed with water, sodium bicarbonate and dried over sodium sulfate. Evaporation of the solvent and chromatography of the residue on an acid-washed alumina column (300 g.) using ether-petroleum ether (v./v. 20-50%) as eluent give the product, methyl-5-methoxy-2-methyl-3-indenyl-α-fluoroacetate.

(B) (Z) α-Fluoro-5-methoxy-2-methyl-(4-pyridinylidene)-3-(N-benzyl)indenylacetamide

In accordance with the procedures described in Example 1, parts E-G, this compound is obtained substituting methyl-5-methoxy-2-methyl-3-indenyl-α-fluoroacetate for 5-fluoro-2-methylindenyl-3-acetic acid in Example 1, part E.

For the introduction of the ═CH-Y part in Scheme III, any of the appropriate heterocyclic aldehydes may be used either directly in the base-catalyzed condensation or in a Wittig reaction in an alternative route. The aldehydes that may be used are listed in Table I below:

TABLE 1
pyrrol-2-aldehyde*
pyrimidine-2-aldehyde
6-methylpyridine-2-aldehyde*
1-methylbenzimidazole-2-aldehyde
isoquinoline-4-aldehyde
4-pyridinecarboxaldehyde*
3-pyridinecarboxaldehyde*
2-pyridinecarboxaldehyde*
4,6-dimethyl-2-pyridinecarboxaldehyde*
4-methyl-pyridinecarboxaldehyde*
4-quinolinecarboxaldehyde*
3-quinolinecarboxaldehyde*
2-quinolinecarboxaldehyde*
2-chloro-3-quinolinecarboxaldehyde*
pyrazinealdehyde
(Prepared as described by Rutner et al., JOC 1963, 28, 1898-99)
pyridazine-3-aldehyde
(Prepared as described by Heinisch et al., Monatshefte Fuer Chemie 108,
213-224,1977)
pyrimidine-4-aldehyde
(Prepared as described by Bredereck et al., Chem. Ber. 1964, 97,
3407-17)
2-methyl-pyrimidine-4-aldehyde
(Prepared as described by Bredereck et al., Chem. Ber. 1964, 97,
3407-17)
pyridazine-4-aldehyde
(Prepared as described by Heinisch et al., Monatshefte Fuer Chemie 104,
1372-1382
(1973))
1-methylindole-3-carboxaldehyde*
1-acetyl-3-indolecarboxaldehyde*
*Available from Aldrich

The aldehydes above can be used in the reaction schemes above in combination with various appropriate amines to produce compounds with the scope of this invention. Examples of appropriate amines are those listed in Table 2 below:

TABLE 2
benzylamine
2,4-dimethoxybenzylamine
2-methoxybenzylamine
2-fluorobenzylamine
4-dimethylaminobenzylamine
4-sulfonaminobenzylamine
1-phenylethylamine (R-enantiomer)
2-amino-2-phenylethanol (S-enantiomer)
2-phenylglycinonitrile (S-enantiomer)

### EXAMPLE 38

(Z)-5-Fluoro-2-Methyl-(4-Pyridylidene)-3-(N-Benzyl) Indenylacetamide Hydrochloride

(Z)-5-Fluoro-2-methyl-(4-pyridylidene)-3-(N-benzyl)indenylacetamide (1396 g; MW 384.45; 3.63 mol) from Example 1 is dissolved at 45° C. in ethanol (28 L). Aqueous HCl (12 M; 363 mL) is added stepwise. The reaction mixture is heated under reflux for 1 hour, is allowed to cool to room temperature, then stored at −10° C. for 3 hours. The resulting solid is filtered off, is washed with ether (2×1.5 L) and is air-dried overnight. Drying under vacuum at 70° C. for 3 days gives (Z)-5-fluoro-2-methyl-(4-pyridylidene)-3-(N-benzyl)indenylacetamide hydrochloride with a melting point of 207-209° C. (R₁═F, R₂═CH₃, R₃═H, R₄═H, R₅═H, R₆═H, R₇═H, n=1, m=1, Y=4-pyridinyl. hydrochloride). Yield: 1481 g ( 97%; 3.51 mol); MW: 420.91 g/mol.

¹H-NMR (DMSO-d₆): 2.18 (s,3,=C—CH₃); 3.54 (s,2,=CH₂CO); 4.28 (d,2,NCH₂); 6.71 (m,1,ar.); 7.17 (m,8,ar.); 8.11 (d,2,ar., AB system); 8.85 (m,1,NH); 8.95 (d,2,ar.,AB system); IR (KBr): 3432 NH; 1635 C═O; 1598 C═C.

### EXAMPLE 39

### (Z)-5-fluoro-2-methyl-(4-pyridylidene)-3-(N-benzyl)-indenylacetamide p-methylbenzenesulfonate

(Z)-5-fluoro-2-methyl-(4-pyridylene)-3-(N-benzyl)indenylacetamide (MW=384.46 g/mol; 5.21 mmol; 2 g) from Example 1 is dissolved in ethanol (50 ml). Solid p-toluenesulfonic acid monohydrate (MW=190.22 g/mol; 5.21 mmol; 991 mg) is added to the stirred solution. The reaction mixture is stirred for 12 hours at room temperature. The ethanol is evaporated in aspirator vacuum. The residue is dried in high vacuum to yield (Z)-5-fluoro-2-methyl-(4-pyridylidene)-3-(N-benzyl)-indenylacetamide p-methylbenzenesulfonate as an orange-red powder.

As to identifying structurally additional PDE2 and PDE5 inhibiting compounds (i.e., non-specific cGMP PDE inhibitors)besides those of Formula I that can be effective therapeutically for the purposes of this invention, one skilled in the art has a number of useful model compounds disclosed herein (as well as their analogs) that can be used as the bases for computer modeling of additional compounds having the same conformations but different chemically. For example, software such as that sold by Molecular Simulations Inc. release of WebLab® ViewerPro™ includes molecular visualization and chemical communication capabilities. Such software includes functionality, including 3D visualization of known active compounds to validate sketched or imported chemical structures for accuracy. In addition, the software allows structures to be superimposed based on user-defined features, and the user can measure distances, angles, or dihedrals.

In this situation, since the structures of active compounds are disclosed above, one can apply cluster analysis and 2D and 3D similarity search techniques with such software to identify potential new additional compounds that can then be screened and selected according to the selection criteria of this invention. These software methods rely upon the principle that compounds, which look alike or have similar properties, are more likely to have similar activity, which can be confirmed using the PDE selection criterion of this invention.

Likewise, when such additional compounds are computer-modeled, many such compounds and variants thereof can be synthesized using known combinatorial chemistry techniques that are commonly used by those of ordinary skill in the pharmaceutical industry. Examples of a few for-hire combinatorial chemistry services include those offered by New Chemical Entities, Inc. of Bothell Washington, Protogene Laboratories, inc., of Palo Alto, Calif., Axys, Inc. of South San Francisco, Calif., Nanosyn, Inc. of Tucson, Ariz., Trega, Inc. of San Diego, Calif., and RBI, Inc. of Natick, Mass. There are a number of other for-hire companies. A number of large pharmaceutical companies have similar, if not superior, in-house capabilities. In short, one skilled in the art can readily produce many compounds for screening from which to select promising compounds for treatment of neoplasia having the attributes of compounds disclosed herein.

To further assist in identifying compounds that can be screened and then selected using the criterion of this invention, knowing the binding of selected compounds to PDE5 and PDE2 protein is of interest. By the procedures discussed below, it is believed that that preferable, desirable compounds meeting the selection criteria of this invention bind to the cGMP catalytic regions of PDE2 and PDE5.

To establish this, a PDE5 sequence that does not include the catalytic domain can be used. One way to produce such a sequence is to express that sequence as a fusion protein, preferably with glutiathione S-transferase (“GST”), for reasons that will become apparent.

RT-PCR method is used to obtain the cGB domain of PDE5 with forward and reverse primers designed from bovine PDE5A cDNA sequence (McAllister-Lucas L. M. et al, J. Biol. Chem. 268, 22863-22873, 1993) and the selection among PDE 1-10 families. 5′-3′, Inc. kits for total RNA followed by oligo (dT) column purification of mRNA are used with HT-29 cells. Forward primer (GAA-TTC-TGT-TAG-AAA-AGC-CAC-CAG-AGA-AAT-G, 203-227) and reverse primer (CTC-GAG-CTC-TCT-TGT-TTC-TTC-CTC-TGC-TG, 1664-1686) are used to synthesize the 1484 bp fragment coding for the phosphorylation site and both low and high affinity cGMP binding sites of human PDE5A (203-1686 bp, cGB-PDE5). The synthesized cGB-PDE5 nucleotide fragment codes for 494 amino acids with 97% similarity to bovine PDE5A. It is then cloned into pGEX-5X-3 glutathione-S-transferase (GST) fusion vector (Pharmacia Biotech) with tac promoter, and EcoRI and XhoI cut sites. The fusion vector is then transfected into E. Coli BL21 (DE3) bacteria (Invitrogen). The transfected BL21 bacteria is grown to log phase, and then IPTG is added as an inducer. The induction is carried at 20° C. for 24 hrs. The bacteria are harvested and lysed. The soluble cell lysate is incubated with GSH conjugated Sepharose 4B (GSH-Sepharose 4B). The GST-cGB-PDE5 fusion protein can bind to the GSH-Sepharose beads, and the other proteins are washed off from beads with excessive cold PBS.

The expressed GST-cGB-PDE5 fusion protein is displayed on 7.5% SDS-PAGE gel as an 85 Kd protein. It is characterized by its cGMP binding and phosphorylation by protein kinases G and A. It displays two cGMP binding sites, and the K_{d} is 1.6±0.2 μM, which is close to K_{d}=1.3 μM of the native bovine PDE5. The GST-cGB-PDE5 on GSH-conjugated sepharose beads can be phosphorylated in vitro by cGMP-dependent protein kinase and cAMP-dependent protein kinase A. The Kₘ of GST-cGB-PDE5 phosphorylation by PKG is 2.7 μM and Vmax is 2.8 μM, while the Kₘ of BPDEtide phosphorylation is 68 μM. The phosphorylation by PKG shows molecular phosphate incorporated into GST-cGB-PDE5 protein on a one-to-one ratio.

A cGMP binding assay for compounds of interest (Francis S. H. et al, J. Biol. Chem. 255, 620-626, 1980) is done in a total volume of 100 μL containing 5 mM sodium phosphate buffer (pH=6.8), 1 mM EDTA, 0.25 mg/mL BSA, H³-cGMP (2 μM, NEN) and the GST-cGB-PDE5 fusion protein (30 μg /assay). Each compound to be tested is added at the same time as ³H-cGMP substrate, and the mixture is incubated at 22° C. for 1 hour. Then, the mixture is transferred to Brandel MB-24 cell harvester with GF/B as the filter membrane followed by 2 washes with 10 mL of cold 5 mM potassium buffer( pH 6.8). The membranes are then cut out and transferred to scintillation vials followed by the addition of 1 mL of H₂O and 6 mL of Ready Safe™ liquid scintillation cocktail to each vial. The vials are counted on a Beckman LS 6500 scintillation counter.

For calculation, blank samples are prepared by boiling the binding protein for 5 minutes, and the binding counts are <1% when compare to unboiled protein. The quenching by filter membrane or other debris are also calibrated.

PDE5 inhibitors, sulindac sulfide, exisulind, E4021 and zaprinast, and cyclic nucleotide analogs, cAMP, cyclic IMP, 8-bromo-cGMP, cyclic UMP, cyclic CMP, 8-bromo-cAMP, 2′-O-butyl-cGMP and 2′-O-butyl-cAMP were selected to test whether they could competitively bind to the cGMP binding sites of the GST-cGB-PDE5 protein. cGMP specifically bound to GST-cGB-PDE5 protein. Cyclic AMP, cUMP, cCMP, 8-bromo-cAMP, 2′-O-butyl-cAMP and 2′-O-butyl-cGMP did not compete with cGMP in binding. Cyclic IMP and 8-bromo-cGMP at high concentration (100 μM) can partially compete with cGMP (2 μM) binding. None of the PDE5 inhibitors showed any competition with cGMP in binding of GST-cGB-PDE5. Therefore, they do not bind to the cGMP binding sites of PDE5.

However, Compound 38 does competitively (with cGMP) bind to PDE 5. Given that Compound 38 does not bind to the cGMP-binding site of PDE5, the fact that there is competitive binding between Compound 38 and cGMP at all means that desirable compounds such as Compound 38 bind to the cGMP catalytic site on PDE5, information that is readily obtainable by one skilled in the art (with conventional competitive binding experiments) but which can assist one skilled in the art more readily to model other compounds. Thus, with the chemical structures of desirable compounds presented herein and the cGMP binding site information, one skilled in the art can model, identify and select (using the selection criteria of this invention) other chemical compounds for use as therapeutics.

### Biological Effects

(A) Cyclooxygenase (COX) Inhibition

COX catalyzes the formation of prostaglandins and thromboxane by the oxidative metabolism of arachidonic acid. The compound of Example 1 of this invention, as well as a positive control, (sulindac sulfide) were evaluated to determine whether they inhibited purified cyclooxygenase Type I (see Table 1 below).

The compounds useful in the practice of this invention were evaluated for inhibitory effects on purified COX. The COX was purified from ram seminal vesicles, as described by Boopathy, R. and Balasubramanian, J., 239:371-377, 1988. COX activity was assayed as described by Evans, A. T., et al., “Actions of Cannabis Constituents on Enzymes Of Arachidonate Metabolism Anti-Inflammatory Potential,” Biochem. Pharmacol., 36:2035-2037, 1987. Briefly, purified COX was incubated with arachidonic acid (100 μM) for 2.0 min at 37° C. in the presence or absence of test compounds. The assay was terminated by the addition of TCA, and COX activity was determined by absorbance at 530 nm.

TABLE I

| | COX I |
|---|---|
| EXAMPLE | % Inhibition(100 μM) |
| Sulindac sulfide | 86 |
| 1 | <25 |

The advantage of very low COX inhibition is that compounds of this invention can be administered to patients without the side effects normally associated with COX inhibition.

(B) cGMP PDE Inhibition

Compounds of this invention are also PDE2 and PDE5 inhibitors as taught in part U.S. patent application Ser. No. 09/046,739 filed Mar. 24, 1998. Compounds can be tested for inhibitory effect on phosphodiesterase activity using either the enzyme isolated from any tumor cell line such as HT-29 or SW-480. Phosphodiesterase activity can be determined using methods known in the art, such as a method using radioactive ³H cyclic GMP (cGMP)(cyclic 3′,5′-guanosine monophosphate) as the substrate for PDE5 enzyme. (Thompson, W. J., Teraski, W. L., Epstein, P. M., Strada, S. J., Advances in Cyclic Nucleotide Research, 10:69-92, 1979, which is incorporated herein by reference). In brief, a solution of defined substrate ³H-cGMP specific activity (0.2 μM; 100,000 cpm; containing 40 mM Tris-HCl (pH 8.0), 5 mM MgCl₂ and 1 mg/ml BSA) is mixed with the drug to be tested in a total volume of 400 μl. The mixture is incubated at 30° C. for 10 minutes with partially purified cGMP-specific PDE isolated from HT-29 cells. Reactions are terminated, for example, by boiling the reaction mixture for 75 seconds. After cooling on ice, 100 μl of 0.5 mg/ml snake venom (O. Hannah venom available from Sigma) is added and incubated for 10 min at 30° C. This reaction is then terminated by the addition of an alcohol, e.g. 1 ml of 100% methanol. Assay samples are applied to a anion chromatography column (1 ml Dowex, from Aldrich) and washed with 1 ml of 100% methanol. The amount of radioactivity in the breakthrough and the wash from the columns in then measured with a scintillation counter. The degree of PDE5 inhibition is determined by calculating the amount of radioactivity in drug-treated reactions and comparing against a control sample (a reaction mixture lacking the tested compound).

Using such protocols, the compound of Example 1 had an IC₅₀ value for PDE5 inhibition of 0.68 μM. Using similar protocols, the compound of Example 38 (“Compound 38”) had an IC₅₀ value for PDE2 of 14 μM, an IC₅₀ value for PDE5 of 4 μM, an IC₅₀ value for PDE1 of 3 μM, and an IC₅₀ value for PDE4 of 6 μM.

(C) Safety Assessment in Mammals

As one skilled in the art will recognize from the data presented below, Compound 38 can safely be given to animals at doses far beyond the tolerable (and in many cases toxic) doses of conventional GSK3 therapies. For example, in an acute toxicity study in rats, single oral doses of Compound 38 administered (in a 0.5% carboxy-methylcellulose vehicle) at doses up to and including 2000 mg/kg resulted in no observable signs of toxicity. At 2000 mg/kg, body weight gains were slightly reduced. A single dose of 1000 mg/kg administered intraperitoneally resulted in reduced body weight gain, with mesenteric adhesions seen in some animals from this group at necropsy.

In dogs, the administration of Compound 38 in capsules at 1000 mg/kg resulted in no signs of toxicity to the single group of two male and two female dogs. Due to the nature of Compound 38 capsules, this dose necessitated the use of at least 13 capsules to each animal, which was judged to be the maximum number without subjecting the animals to stress. Therefore, these dogs were subsequently administered seven consecutive doses of 1000 mg/kg/day. At no time in either dosing phase were any obvious signs of drug-related effects observed.

Thus, on a single-dose basis, Compound 38 is not acutely toxic. Based on the findings of these studies, the oral LD₅₀ of Compound 38 was considered to be greater than 1000 mg/kg in dogs and 2000 mg/kg in rats, and the intraperitoneal LD₅₀ was considered to be greater than 1000 mg/kg in rats.

A seven-day dose-range finding study in rats, where Compound 38 was evaluated by administering it at doses of 0, 50, 500 or 2000 mg/kg/day resulting in no observable signs of toxicity at 50 mg/kg/day. At 500 mg/kg/day, treatment-related effects were limited to an increase in absolute and relative liver weights in female rats. At 2000 mg/kg/day, effects included labored breathing and/or abnormal respiratory sounds, decreased weights gains and food consumption in male rats, and increased liver weights in female rats. No hematological or blood chemistry changes nor any microscopic pathology changes, were seen at any dose level.

A 28-day study in rats was also carried out at 0, 50, 500 and 2000 mg/kg/day. There were no abnormal clinical observations attributed to Compound 38, and body weight changes, ophthalmoscopic examinations, hematological and blood chemistry values and urinalysis examinations were unremarkable. No macroscopic tissue changes were seen at necropsy. Organ weight data revealed statistically significant increase in liver weights at 2000 mg/kg/day, and statistically significant increases in thyroid weights for the 2000 mg/kg/day group. The slight liver and thyroid increases at the lower doses were not statistically significant. Histopathological evaluation of tissues indicated the presence of traces of follicular cell hypertrophy, increased numbers of mitotic figures (suggestive of possible cell proliferation) in the thyroid gland and mild centrilobular hypertrophy in the liver. These changes were generally limited to a small number of animals at the 2000 mg/kg/day dose, although one female at 500 mg/kg/day had increased mitotic figures in the thyroid gland. The findings in the liver may be indicative of a very mild stimulation of liver microsomal enzymes, resulting in increased metabolism of thyroid hormones, which in turn resulted in thyroid stimulation.

A long-term safety assessment study was conducted in rats to investigate Compound 38 at 50, 200 and 500 mg/kg/day following repeated oral dosing for 91 consecutive days. Orally administered Compound 38 did not produce any major toxicological effects in rats. The only finding was a dose-related trend to increased liver and thyroid/parathyroid weights noted in males and females at 200 and 500 mg/kg/day. Microscopically, slight hepatocellular hypertrophy at 200 and 500 mg/kg/day groups, follicular cell hypertrophy at 500 mg/kg/day and increase in accumulation of hyalin droplets in the kidneys at 200 and 500 mg/kg/day group. However, no changes in clinical biochemistry and hematology were evident. These changes were not associated with any gross clinical abnormality.

Dogs were also dosed orally with Compound 38 at 50, 150 and 300 mg/kg/day for 91 consecutive days. There were no toxicological effects in the dog following 91 days of dosing. Orange discoloration of the feces (same color as Compound 38) was seen in the 150 and 300 mg/kg/day groups. This finding suggested that most of Compound 38 was being eliminated via the feces. Slightly lowered body weights were noted in the highest dose group. This dose was also associated with increased liver weights. However, there were no microscopic alterations to support the increase in liver weight. Therefore, we concluded that Compound 38 is well tolerated in the dog.

Finally as to safety, in a single, escalating dose human clinical trial, patients, human safety study in which the drug was taken orally, Compound 38 produced no significant side effects at any dose (i.e., 50 mg BID, 100 mg BID, 200 mg BID and 400 mg BID).—doses above the level believed to be therapeutic for human patients.

One skilled in the art should recognize that any of the side effects observed in these safety studies occurred at very high doses, in excess of recommended human doses and are extremely minimal compared to what one would expect with many other drugs.

Screening Methodologies

To identify new compounds that inhibit GSK3β in the manner of this invention, one can retrace what we have taught in this application. Namely, a compound according to this invention can be found by evaluating its ability to increase the activity of PKG as taught above. Alternatively, the compound can be identified by its ability to inhibit cGMP PDEs non-selectively, (i.e., at least inhibit PDE2 and PDE5. It is also believed to be desirable to inhibit PDE1). As confirmation that a desired compound has been identified, one can then assess whether GSK3p is inhibited, and as further confirmation that β-catenin does not accumulate when the drug is exposed to the cells in question. These procedures are described above, and individually, but not in combination, known in the art.

To avoid a side effect that may or may not be desired, one can then assess whether the compound inhibits a cyclooxygenase enzyme. These methodologies are known as well.

As one skilled in this art will appreciate, there are various ways to assess whether a compound inhibits, activates, modifies (e.g., phosphorylates) etc. a particular protein target. We do not mean to suggest that the specific tests we have described herein are the only such tests. Other methods of covalent modification, for example, would suffice.

We claim

1. A method of selecting a compound for treatment of a disease where GSK3β is desired, comprising:
(a) evaluating whether the compound increases PKG activity;
(b) evaluating whether the compound inhibits GSK3β; and
(c) selecting the compound that causes an increase in PKG activity and inhibits GSK3β.

2. The method of claim 1 further comprising evaluating whether the compound inhibits cGMP PDE, and selecting the compound that inhibits cGMP PDE.

3. The method of claim 1 further comprising evaluating whether the compound causes β-catenin to accumulate in the cells of the type to be treated, and selecting the compound that so does not cause β-catenin to accumulate.

4. The method of claim 1 further comprising evaluating whether the compound inhibits cGMP-specific phosphodiesterase (“PDE”) and selecting the compound that inhibits said PDE.

5. The method of claim 1 further comprising evaluating whether the compound increases PKG expression, and selecting the compound if it increases PKG expression.

6. The method of claim 1 further comprising evaluating whether the compound increases PKG activation, and selecting the compound if it increases PKG activation.

7. The method of claim 1 further comprising:
determining the cyclooxygenase (COX) inhibitory activity of the compound; and
selecting the compound with COX inhibitory activity lower than its activity for increasing PKG activity.

8. A method of inhibiting GSK3β in non-neoplastic mammalian cells, comprising increasing the activity of PKG in said cells.

9. The method of claim 8 wherein PKG activity is increased by inhibiting the cGMP PDE activity in said cells.

10. A method of inhibiting GSK3β in non-neoplastic mammalian cells, comprising increasing the activity of PKG in said cells without substantially inhibiting COX.

11. The method of claim 10 wherein PKG activity is increased by inhibiting the cGMP PDE activity in said cells.

12. A method of inhibiting GSK3β in non-neoplastic mammalian cells, comprising increasing the activity of PKG in said cells continuously over an extended period of time.

13. A method of inhibiting GSK3β in non-neoplastic mammalian cells, comprising increasing the activity of PKG in said cells without exposing said cells to exisulind.

14. The method of claim 13 wherein PKG activity is increased by inhibiting the cGMP PDE activity in said cells.

15. A method of inhibiting GSK3β in non-neoplastic mammalian cells, comprising increasing the activity of PKG in said cells without substantially inhibiting COX and without exposing said cells to exisulind.

16. The method of claim 15 wherein PKG activity is increased by inhibiting the cGMP PDE activity in said cells.

17. A method of inhibiting GSK3β in non-neoplastic mammalian cells, comprising increasing the activity of PKG in said cells continuously over an extended period of time without exposing said cells to exisulind.
Qqq

18. A method of inhibiting GSK3β in non-neoplastic mammalian cells, comprising increasing the activity of PKG in said cells without increasing PKB/Akt kinase activity.

19. The method of claim 18 wherein PKG activity is increased by inhibiting the cGMP PDE activity in said cells.

20. A method of inhibiting GSK3β in non-neoplastic mammalian cells, comprising increasing the activity of PKG in said cells without substantially inhibiting COX and without substantially increasing PKB/Akt kinase activity.

21. The method of claim 20 wherein PKG activity is increased by inhibiting the cGMP PDE activity in said cells.

22. A method of inhibiting GSK3β in non-neoplastic mammalian cells, comprising increasing the activity of PKG in said cells continuously over an extended period of time without substantially increasing PKB/Akt kinase activity.

23. A method of inhibiting GSK3β in non-neoplastic mammalian cells, comprising increasing the activity of PKG in said cells without exposing said cells to exisulind and without substantially increasing PKB/Akt kinase activity.

24. The method of claim 23 wherein PKG activity is increased by inhibiting the cGMP PDE activity in said cells.

25. A method of inhibiting GSK3β in non-neoplastic mammalian cells, comprising increasing the activity of PKG in said cells without substantially inhibiting COX or increasing PKB/Akt kinase activity and without exposing said cells to exisulind.

26. The method of claim 25 wherein PKG activity is increased by inhibiting the cGMP PDE activity in said cells.

27. A method of inhibiting GSK3β in non-neoplastic mammalian cells, comprising increasing the activity of PKG in said cells continuously over an extended period of time without exposing said cells to exisulind and without substantially increasing PKB/Akt kinase activity.

23. A method of inhibiting GSK3β in type II diabetic non-neoplastic mammalian cells, comprising increasing the activity of PKG in said cells without exposing said cells to exisulind and without substantially increasing PKB/Akt kinase activity.
